(19) United States
(12) Patent Application Publication
(10) Pub. No.: US 2023/0174460 A1
(43) Pub. Date: Jun. 8, 2023

(54) AFMT ANALOGS AND THEIR USE IN METHODS OF TREATING PARKINSON'S DISEASE

(71) Applicant: President and Fellowes of Harvard College, Cambridge, MA (US)

(72) Inventors: Emily Balskus, Somerville, MA (US); Chi Le, Boston, MA (US)

(21) Appl. No.: 17/920,588

(22) PCT Filed: Apr. 21, 2021
(86) PCT No.: PCT/US2021/028492

§ 371 Date: Oct. 21, 2022

Related U.S. Application Data
(60) Provisional application No. 63/013,270, filed on Apr. 21, 2020.

(51) Int. Cl. C07C 229/42; C07D 261/04; C07D 239/34; C07D 241/18; C07D 239/42; C07D 237/14; C07D 231/56; C07D 213/65; C07C 229/36; C07D 213/64; C07C 291/04

(52) U.S. Cl. CPC C07C 229/42; C07D 261/04; C07D 239/34; C07D 241/18; C07D 239/42; C07D 237/14; C07D 231/56; C07D 213/65; C07C 229/36; C07D 213/64; C07C 291/04

(57) ABSTRACT

The present disclosure provides compounds of formula (I), (II), and (Ia):

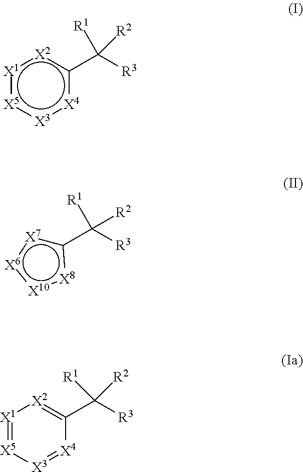

Methods of preparing these molecules and their use for treatment of Parkinson's Disease are described.

### CROSS-REFERENCE TO RELATED APPLICATIONS

This application claims the benefit of, and priority to, U.S. Patent Application No. 63/013,270, filed Apr. 21, 2020, the disclosure of which is hereby incorporated by reference herein in its entirety.

### BACKGROUND

The trillions of organisms that inhabit the human gut (the human gut microbiota) can modify the structures of ingested compounds, including drugs, in a manner distinct from host cells. Such transformations can be critical for drug efficacy or be the source of toxicity. There is also often substantial inter-individual variation in gut microbial drug metabolism between different patients. Though >60 examples of gut microbial drug metabolism are known, very few of these activities have been linked to specific gut microbes, genes, and enzymes. This highlights a critical need to uncover the molecular basis for microbiota-drug interactions in order to facilitate discovering the organisms and genes responsible for these transformations and inform strategies for improving drug efficacy via manipulation of microbial metabolic activities.

One prominent example of gut microbial metabolism altering drug efficacy is the transformation of the front-line Parkinson's disease medication levodopa (L-dopa). L-dopa is prescribed to manage motor symptoms resulting from dopaminergic neuron loss in the substantia nigra. After crossing the blood-brain barrier, it is decarboxylated by aromatic amino acid decarboxylase (AADC) to give dopamine, the active therapeutic agent. However, dopamine generated in the periphery cannot cross the blood-brain barrier, and only 1-5% of L-dopa reaches the brain due to extensive pre-systemic metabolism in the gut by enzymes including AADC. In addition to reducing drug availability, peripheral production of dopamine also causes gastrointestinal side effects, can lead to orthostatic hypotension through activation of vascular dopamine receptors, and may include cardiac arrhythmias. To decrease this unwanted metabolism, L-dopa is co-administered with AADC inhibitors such as carbidopa. Despite this, ˜60% of L-dopa is still metabolized peripherally and patients display significant variability in response to the drug, including loss of efficacy over time. Thus, there is a clear, unmet opportunity to improve patient response to L-dopa by preventing this peripheral metabolism.

### SUMMARY

The invention relates, in part, to compounds that inhibit gut microbial L-dopa metabolism, compositions comprising such compounds, and methods of using such compounds and compositions.

Provided herein are compounds of formula (I) or formula (II):

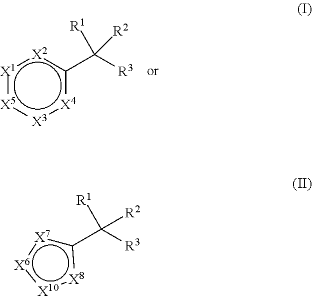

or a pharmaceutically acceptable salt thereof, wherein, as permitted by valence and stability:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or —C(R¹¹)═;
X³ is —N═ or —C(R⁸)—
X⁴ is ═N— or ═C(R¹²)—;
provided that
   no more than two of X¹ to X⁵ are ═N—; and
   when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R⁸)—;
X¹⁰ is ═N—, —NR¹³—, or ═C(R⁴)—;
provided that, if at least three of X⁶ to X¹⁰ are ═N—, one of X⁶ to X¹⁰ is —NR¹³—;
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂ or

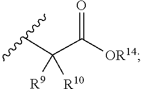

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, —NHBoc, —ONH₂, or —NHNH₂;
or R² and R³, together with the carbon atom to which they are attached, combine to form

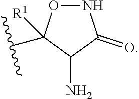

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or

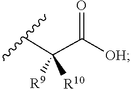

and
R¹⁰ is amino, —ONH₂, or —NHNH₂.

Further provided herein are compounds of formula (Ia):

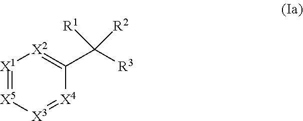

or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or

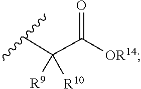

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

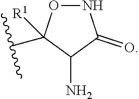

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or

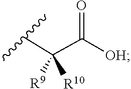

and
R¹⁰ is amino, —ONH₂, or —NHNH₂.

In some embodiments, the compound of Formula I, Ia, or II is not:

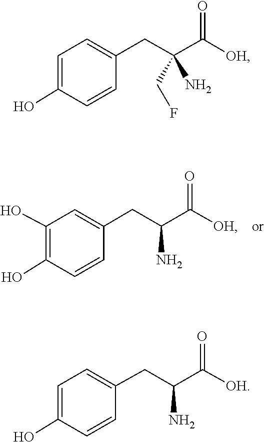 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

In some such embodiments, the compound of Formula I, Ia, or II is not

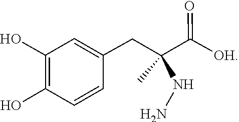 [SMILES: C[C@@](Cc1ccc(O)c(O)c1)(NN)C(=O)O]

Some embodiments of the invention relate to a pharmaceutical composition comprising a compound of formula I, Ia, or II, or a pharmaceutically acceptable salt, biologically active metabolite, solvate, hydrate, prodrug, enantiomer or stereoisomer thereof, and one or more pharmaceutically acceptable carriers, alone or in combination with another therapeutic agent. Such pharmaceutical compositions of the invention can be administered in accordance with a method of the invention, typically as part of a therapeutic regimen for treatment or prevention of conditions and disorders related to Parkinson's Disease.

Certain embodiments of the invention relate to a method of inhibiting a tyrosine decarboxylase (TyrDC), comprising contacting the TyrDC with a compound of formula (I), (Ia), or (II), or a pharmaceutically acceptable salt thereof.

Certain embodiments of the invention relate to a method of treating Parkinson's Disease in a subject in need thereof, comprising administering to a subject in need thereof an effective amount (e.g., a therapeutically effective amount) of one or more compounds, or pharmaceutically acceptable salts thereof, or pharmaceutical compositions of the invention.

### BRIEF DESCRIPTION OF THE FIGURES

FIG. 1 shows a schematic for methods of inhibiting TyrDC according to some embodiments of the present disclosure.

FIGS. 2A-2C show the optimization strategy for inhibitors of microbial L-dopa decarboxylation according to some embodiments of the present disclosure.

FIGS. 3A-3C show data assessing the reactivity of tyrosine analog compounds according to some embodiments of the present disclosure. FIG. 3A shows the results of whole-cell decarboxylation activity of compounds according to some embodiments of the present disclosure. FIG. 3B shows a comparison of inhibition of whole-cell L-dopa decarboxylation for benzyl aminooxy compounds according to some embodiments of the present disclosure. FIG. 3C shows EC₅₀ data for benzyl aminooxy compounds according to some embodiments of the present disclosure.

FIG. 4 shows data on decarboxylation of inhibitors by E. faecalis MMH594 WT according to some embodiments of the present disclosure.

FIG. 5 shows data from an assay of inhibitory effect on L-dopa decarboxylation in E. faecalis whole cells according to some embodiments of the present disclosure.

FIG. 6 shows a schematic for mouse experiments to examine the efficacy of AFMT against L-dopa decarboxylation by E. faecalis.

### DETAILED DESCRIPTION

L-dopa is an important Parkinson's disease medication which must enter the brain to be effective, and it is well established that microbial metabolism of this drug in the human gut negatively affects drug availability and can lead to side effects. Inhibition of this microbial gut metabolism has been proposed as a means of improving response to L-dopa in Parkinson's disease patients.

### Definitions

For convenience, certain terms employed in the specification, examples, and appended claims are collected here. All definitions, as defined and used herein, supersede dictionary definitions, definitions in documents incorporated by reference, and/or ordinary meanings of the defined terms.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

The term “acyl” is art-recognized and refers to a group represented by the general formula hydrocarbyl-C(O)—, preferably alkyl-C(O)—.

The term “acylamino” is art-recognized and refers to an amino group substituted with an acyl group and may be represented, for example, by the formula hydrocarbyl-C(O)NH—.

The term “acyloxy” is art-recognized and refers to a group represented by the general formula hydrocarbyl-C(O)O—, preferably alkyl-C(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and from 1 to about 20 carbon atoms, preferably from 1 to about 10 carbon atoms unless otherwise defined, and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. A C₂-C₆ alkenyl group is also referred to as a “lower alkenyl” group.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include, but are not limited to, methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, an oxo, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituent on substituted alkyls are selected from C₁-C₆ alkyl, C₃-C₆ cycloalkyl, halogen, carbonyl, cyano, or hydroxyl. In more preferred embodiments, the substituents on the substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “Cₓ-C_{y}”, when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C₂-C_{y}alkenyl” and “C₂-C_{y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

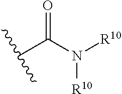

wherein each R¹⁰ independently represent a hydrogen or hydrocarbyl group, aryl, heteroaryl, acyl, or alkoxy, or two R¹⁰ are taken together with the N atom to which they are attached complete a heterocycle having from 3 to 8 atoms in the ring structure.

The terms “amine” and “amino” are art-recognized and refer to both unsubstituted and substituted amines and salts thereof, e.g., a moiety that can be represented by

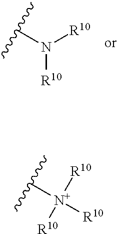

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- to 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, aniline, and the like. Example substitutions on an aryl group include a halogen, a haloalkyl such as trifluoromethyl, a hydroxyl, a carbonyl (such as a carboxyl, an alkoxycarbonyl, a formyl, or an acyl such as an alkylC(O)), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

The term “carbamate” is art-recognized and refers to a group

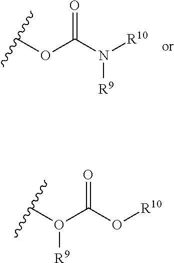

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The term “carbocycle” refers to a saturated or unsaturated ring in which each atom of the ring is carbon. The term carbocycle includes both aromatic carbocycles and non-aromatic carbocycles. Non-aromatic carbocycles include both cycloalkyl and cycloalkenyl rings. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with an carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

A “cycloalkyl” group is a cyclic hydrocarbon, which is completely saturated. “Cycloalkyl” includes monocyclic and bicyclic rings. Typically, a monocyclic cycloalkyl group has from 3 to about 10 carbon atoms, from 3 to 8 carbon atoms, or more typically from 3 to 6 carbon atoms, unless otherwise defined. The second ring of a bicyclic cycloalkyl may be selected from saturated, unsaturated and aromatic rings. Cycloalkyl includes bicyclic substituents in which one, two or three or more atoms are shared between the two rings (e.g., fused bicyclic substituents, bridged bicyclic substituents, and spirocyclic substituents).

The term “fused bicyclic substituent” refers to a bicyclic substituent in which two rings share two adjacent atoms. In other words, the rings share one covalent bond, i.e., the so-called bridgehead atoms are directly connected. For example, in a fused cycloalkyl, each of the rings shares two adjacent atoms with the other ring, and the second ring of a fused bicyclic cycloalkyl may be selected from saturated, unsaturated and aromatic rings.

A “cycloalkenyl” group is a cyclic hydrocarbon containing one or more double bonds.

The term “bridged bicyclic substituent” refers to a bicyclic substituent in which the two rings share three or more atoms, separating the two bridgehead atoms by a bridge containing at least one atom. For example, norbornanyl, also known as bicyclo[2.2.1]heptanyl, can be thought of as a pair of cyclopentane rings each sharing three of their five carbon atoms. The term “spirocyclic substituent” refers to a bicyclic substituent in which the two rings have only one single atom, the spiro atom, in common.

The term “diazo”, as used herein, refers to a group represented by the formula ═N═N.

The term “disulfide” is art-recognized and refers to a group —S—S—R^{A}, wherein R^{A} represents a hydrocarbyl group.

The term “enol ester”, as used herein, refers to a group —C(O)O—C(R^{A})═C(R^{A})₂ wherein R^{A} represents a hydrocarbyl group.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “heteroalkyl”, as used herein, refers to a saturated or unsaturated chain of carbon atoms and at least one heteroatom, for example, wherein no two heteroatoms are adjacent.

The terms “heteroaralkyl” and “hetaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include 5- to 10-membered cyclic or polycyclic ring systems, such as pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, preferably 3- to 7-membered rings, more preferably 5- to 6-membered rings, in some instances, most preferably a 5-membered ring, in other instances, most preferably a 6-membered ring, which ring structures include at least one heteroatom, preferably one to four heteroatoms, more preferably one or two heteroatoms. The terms “heterocyclyl” and “heterocyclic” also include 5- to 10-membered polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, pyrrolidine, piperidine, piperazine, pyrrolidine, tetrahydropyran, tetrahydrofuran, morpholine, lactones, lactams, oxazoles, imidazolines, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer, more preferably three or fewer. A “lower alkyl”, for example, refers to an alkyl group that contains ten or fewer carbon atoms, preferably six or fewer, more preferably three or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “orthoester” as used herein is art-recognized and refers to a group —C(OR^{A})₃, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “phosphoester”, as used herein, refers to a group —OP(═O)(OH)₂.

The term “phosphodiester”, as used herein, refers to a group —OP(═O)(OR^{A})₂ wherein R^{A} represents a hydrocarbyl group.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “pseudohalide,” as used herein, means a polyatomic analogue of a halide. Preferred pseudohalides include sulfonates, e.g., arylsulfonates and alkylsulfonates, such as p-toluenesulfonate and trifluoromethanesulfonate.

The term “selenide”, as used herein, is equivalent to an ether, wherein the oxygen is replaced with a selenium.

The term “selenoxide” is art-recognized and refers to the group —Se(O)—R^{A}, wherein R^{A} represents a hydrocarbyl.

The term “siloxane” is art-recognized and refers to a group with an Si—O—Si linkage, such as the group —Si(R^{A})₂—O—Si—(R^{A})₃, wherein each R^{A} independently represents hydrogen or hydrocarbyl, such as alkyl, or two R^{A} taken together with the intervening atom(s) complete a heterocycle having from 4 to 8 atoms in the ring structure.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, an oxo, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an alkyl, an amino, an amido, an amidine, an imine, an oxime, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁-C₆ alkyl, C₃-C₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

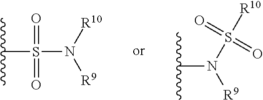

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂-R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

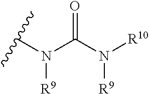

wherein each R⁹ and R¹⁰ independently represents hydrogen or a hydrocarbyl, such as alkyl, or either occurrence of R⁹ taken together with R¹⁰ or the other R⁹ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, anisoyl (“An”), benzyl (“Bn”), benzoyl (“Bz”), benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt that is suitable for or compatible with the treatment of patients.

The term “pharmaceutically acceptable acid addition salt” as used herein means any non-toxic organic or inorganic salt of any base compounds disclosed herein. Illustrative inorganic acids that form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the invention for laboratory use, or for subsequent conversion to a pharmaceutically acceptable acid addition salt.

The term “pharmaceutically acceptable basic addition salt” as used herein means any non-toxic organic or inorganic base addition salt of any acid compounds of the invention, or any of their intermediates. Illustrative inorganic bases that form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

The invention also includes various isomers and mixtures thereof “Isomer” refers to compounds that have the same composition and molecular weight but differ in physical and/or chemical properties. The structural difference may be in constitution (geometric isomers) or in the ability to rotate the plane of polarized light (stereoisomers).

Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by ““which represents””, ““or””.

“R,” “S,” “S*,” “R*,” “E,” “Z,” “cis,” and “trans,” indicate configurations relative to the molecule having unspecified stereochemistry.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the geometry of a disclosed compound is named or depicted by structure, the named or depicted geometrical isomer is at least 60%, 70%, 80%, 90%, 99% or 99.9% by weight pure relative to the other geometrical isomers.

When a disclosed compound is named or depicted by structure without indicating the stereochemistry, and the compound has at least one chiral center, it is to be understood that the name or structure encompasses one enantiomer of the compound free from the corresponding optical isomer, a racemic mixture of the compound and mixtures enriched in one enantiomer relative to its corresponding optical isomer.

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “prodrug” means a pharmacological derivative of a parent drug molecule that requires biotransformation, either spontaneous or enzymatic, within the organism to release the active drug. For example, prodrugs are variations or derivatives of the compounds of the invention that have groups cleavable under certain metabolic conditions, which when cleaved, become the compounds of the invention. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form. Prodrug forms often offer advantages of solubility, tissue compatibility, or delayed release in the mammalian organism (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability.

As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds having a carbonate, carbamate, amide or alkyl ester moiety covalently bonded to any of the above substituents disclosed herein.

A “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein.

### Compounds

Provided herein are compounds of formula (I) and formula (II):

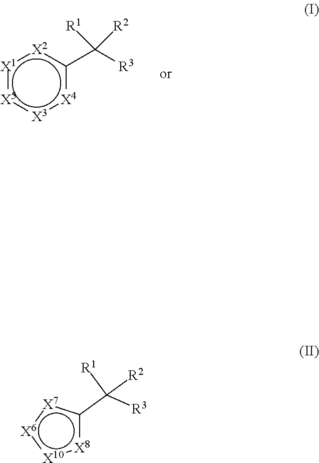

or a pharmaceutically acceptable salt thereof, wherein, as permitted by valence and stability:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or —C(R¹¹)═;
X³ is —N═ or —C(R⁸)—
X⁴ is ═N— or ═C(R¹²)—;
provided that
   no more than two of X¹ to X⁵ are ═N—; and
   when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R′)—;
X¹⁰ is ═N—, —NR¹³—, or ═C(R⁴)—;
provided that, if at least three of X⁶ to X¹⁰ are ═N—, one of X⁶ to X¹⁰ is —NR¹³—;
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂ or

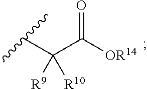

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

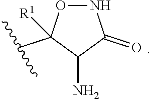

In some embodiments, R¹⁴ is, independently at each occurrence, H or CH₃.

In some embodiments, R³ is

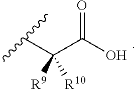

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or

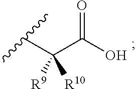

and
R¹⁰ is amino, —ONH₂, or —NHNH₂.

In some embodiments, the compound of Formula (I) or Formula (II) is not:

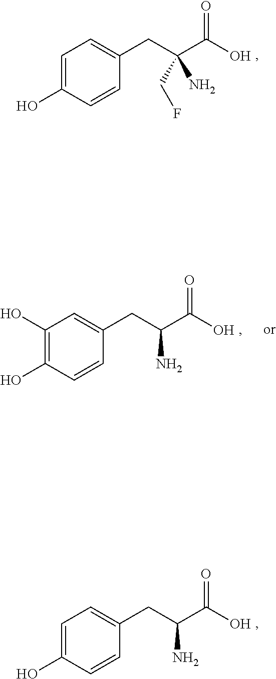 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

or a pharmaceutically acceptable salt of any of the foregoing. In some such embodiments, the compound of Formula (I) or Formula (II) is not

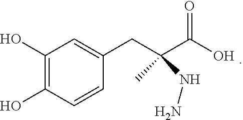 [SMILES: C[C@@](Cc1ccc(O)c(O)c1)(NN)C(=O)O]

In some embodiments, R³ is

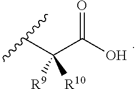

In some embodiments, X¹ is CR⁷, X² is CR¹¹, X³ is CR⁸, X⁴ is CR¹², and X⁵ is CR⁴; or X⁶ is CR⁷, X⁷ is CR¹¹, X⁸ is CR⁸, and X¹⁰ is CR⁴. In some such embodiments, R⁷ is H and R⁸ is H. In other such embodiments, R⁷ is hydroxyl or halogen. In still other such embodiments, R¹¹ is halogen, such as fluorine or chlorine.

In some embodiments, X⁵ is CR⁴ and R⁴ is hydroxyl; or X¹⁰ is CR⁴ and R⁴ is hydroxyl. In certain such embodiments, X³ is —N═.

In other embodiments, X⁵ is CR⁴ and R⁴ is C₁₋₆ alkyl optionally substituted with one or more halogen, such as CF₃, CF₂H, or CFH₂.

In some embodiments, R¹ is H and R² is H.

In some embodiments, R⁷ is hydroxyl, halogen, or amino, such as hydroxyl or halogen.

In some embodiments, X¹ is ═N—. In some embodiments, X² is —N═. In some embodiments, X³ is —N═. In some embodiments, X⁴ is ═N—.

In some embodiments, R⁹ is CFH₂, CF₂H, or CH₃. In some such embodiments, R¹⁰ is —NHNH₂. In certain preferred such embodiments, R⁹ is CFH₂ or CF₂H.

In some embodiments, the compound is:

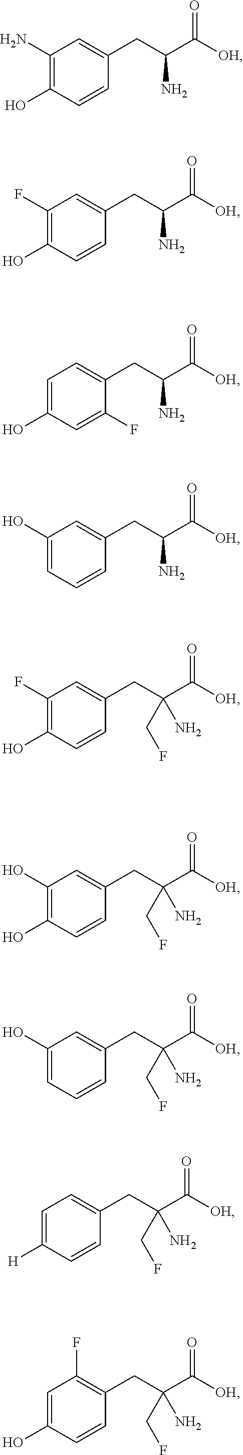

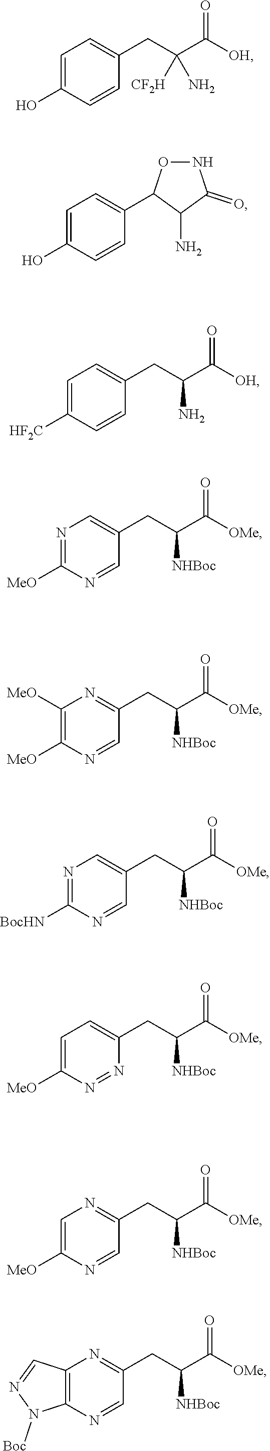

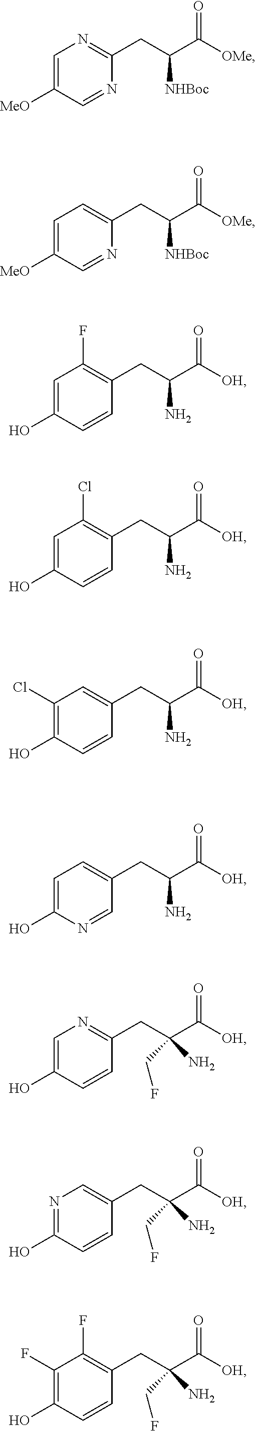

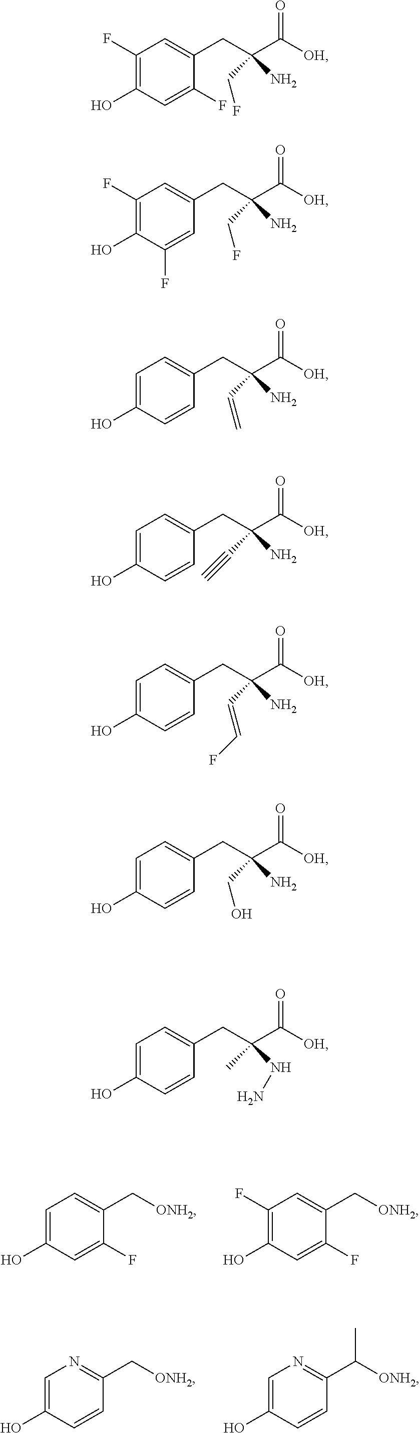

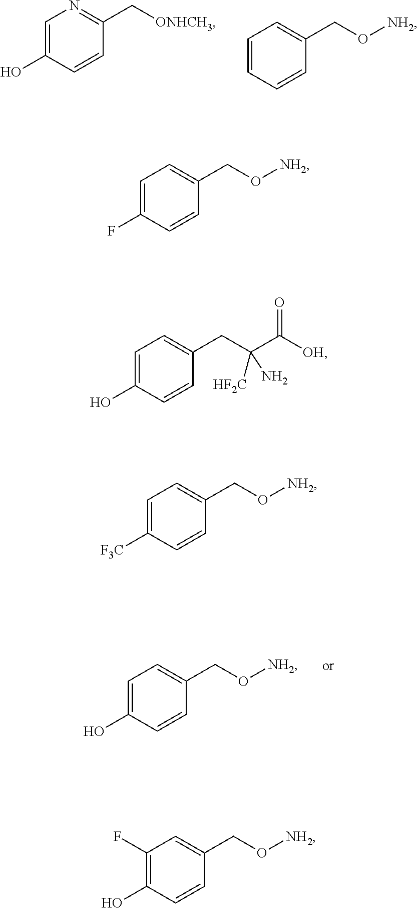

or a pharmaceutically acceptable salt thereof. In some such embodiments, the compound is

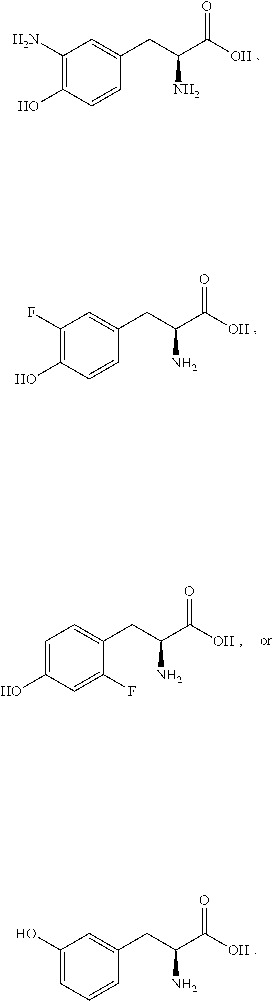 [SMILES: Nc1cc(C[C@H](N)C(=O)O)ccc1O ; N[C@@H](Cc1ccc(O)c(F)c1)C(=O)O ; N[C@@H](Cc1ccc(O)cc1F)C(=O)O ; N[C@@H](Cc1cccc(O)c1)C(=O)O]

In other such embodiments, the compound is:

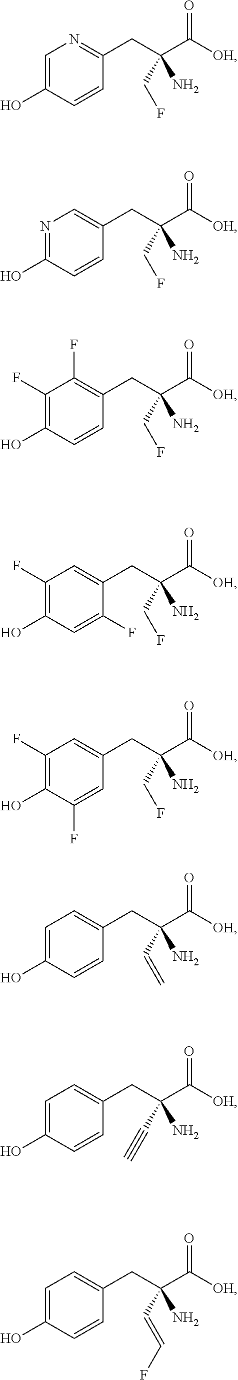

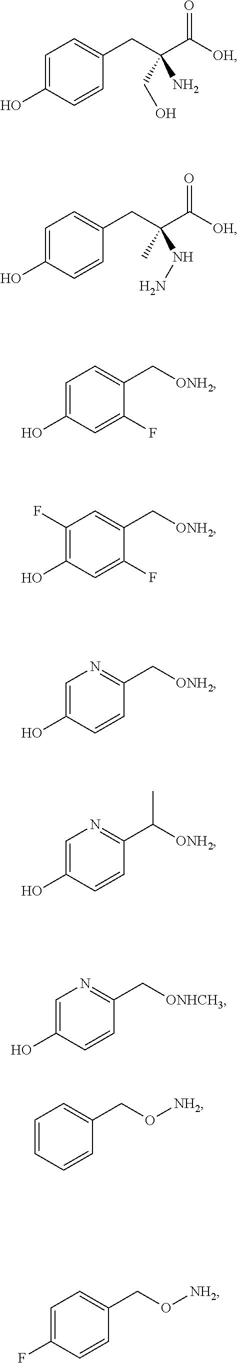

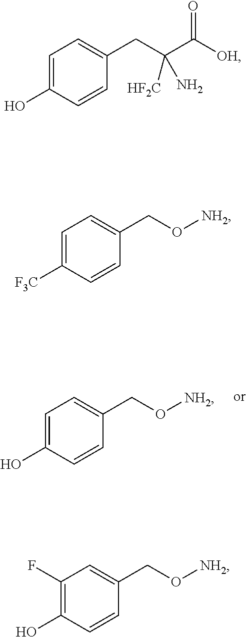

or a pharmaceutically acceptable salt thereof.

In some embodiments, the compound is a compound of formula (I).

In some embodiments, —N(R⁶)C(O)R⁵ is -NHBoc. In some embodiments, R⁴ is halogen, such as F or Cl. In other embodiments, R⁴ is C₁₋₆ alkyl, optionally substituted with one or more halogen, such as CH₃, CF₃, CF₂H, or CFH₂.

Also provided herein is a compound of formula (Ia):

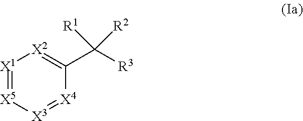

or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or

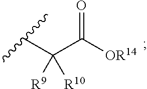

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

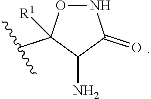

In some embodiments, R¹⁴ is, independently at each occurrence, H or CH₃. In some embodiments, R³ is

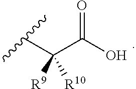

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or

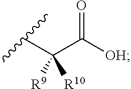

and
R¹⁰ is amino, —ONH₂, or —NHNH₂.

In some embodiments, the compound is not

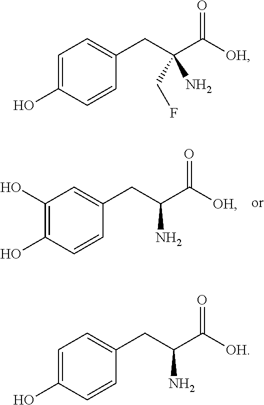

In some such embodiments, the compound is not

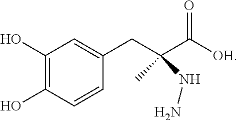 [SMILES: C[C@@](Cc1ccc(O)c(O)c1)(NN)C(=O)O]

In some embodiments, R³ is

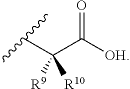

In some embodiments, X¹ is CR⁷, X² is CR¹¹, X³ is CR⁸, X⁴ is CR¹², and X⁵ is CR⁴. In some such embodiments, R⁷ is H and R⁸ is H. In other such embodiments, R⁷ is hydroxyl or halogen. In still other such embodiments, R¹¹ is halogen, such as fluorine or chlorine.

In some embodiments, X⁵ is CR⁴ and R⁴ is hydroxyl. In certain such embodiments, X³ is —N═.

In some embodiments, R¹ is H and R² is H.

In some embodiments, R⁷ is hydroxyl, halogen, or amino, such as hydroxyl or halogen.

In some embodiments, X¹ is ═N—. In some embodiments, X² is —N═. In some embodiments, X³ is —N═. In some embodiments, X⁴ is ═N—.

In some embodiments, R⁹ is CFH₂, CF₂H, or CH₃. In some such embodiments, R¹⁰ is —NHNH₂. In certain preferred such embodiments, R⁹ is CFH₂ or CF₂H.

In some embodiments, the compound is:

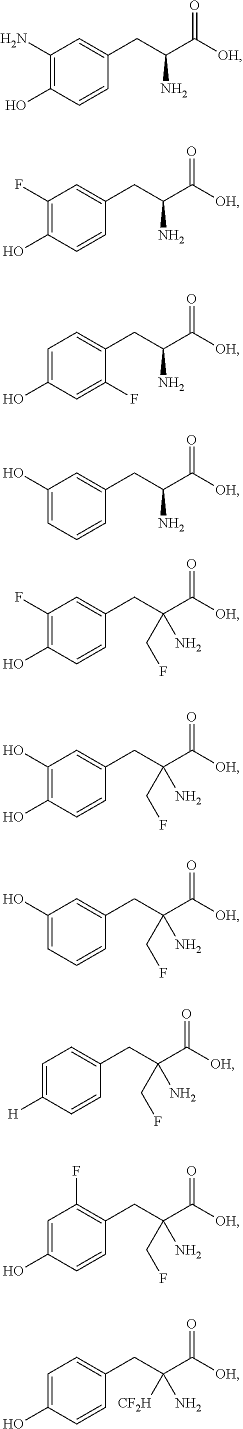

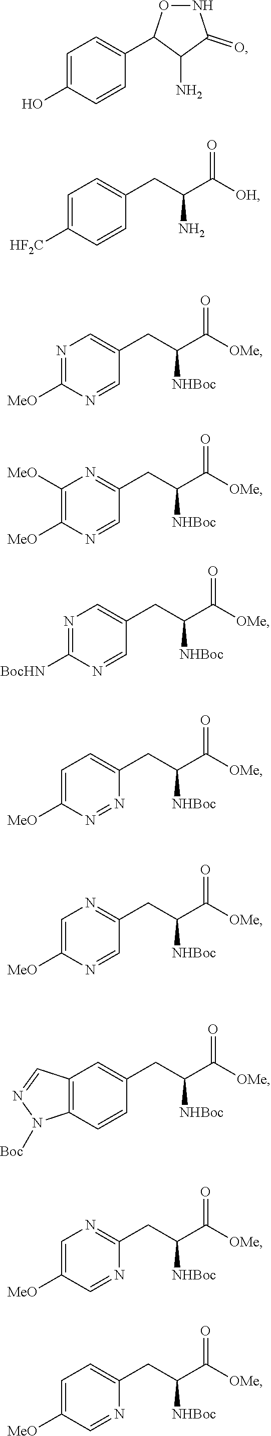

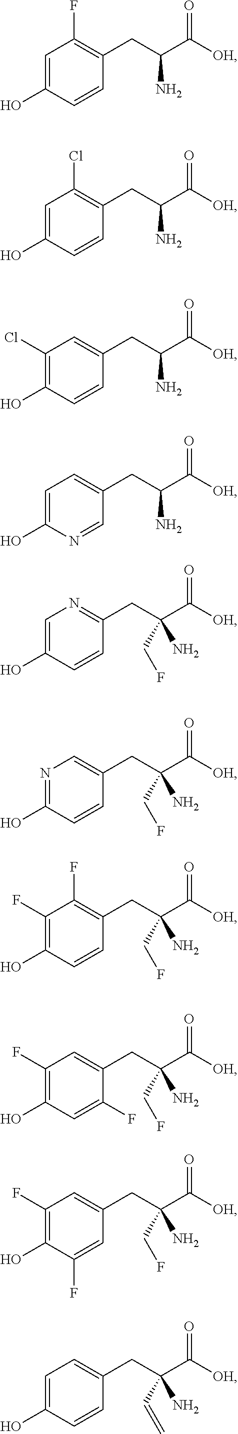

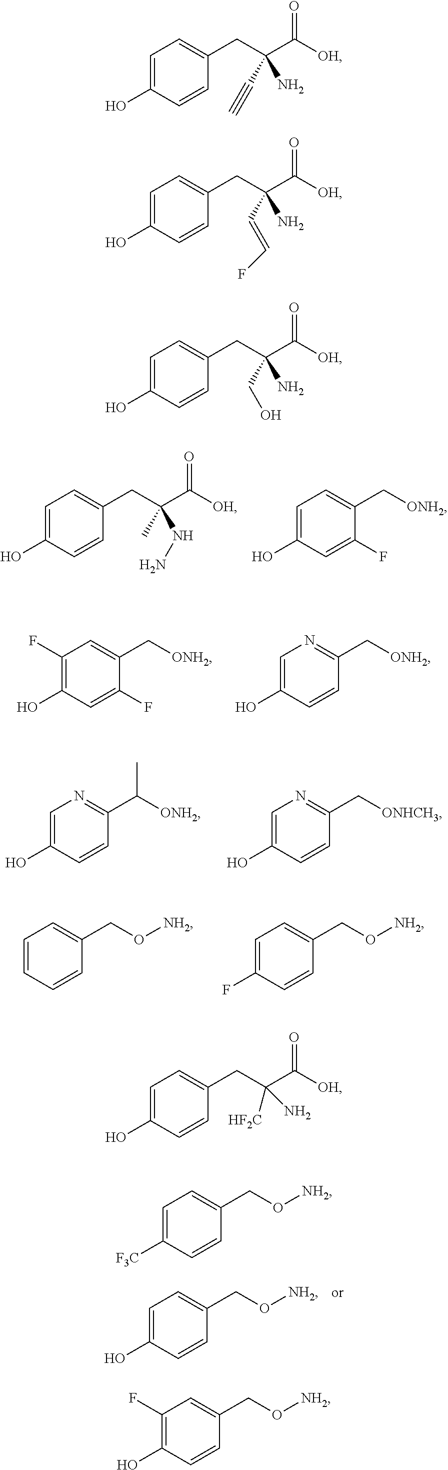

or a pharmaceutically acceptable salt thereof. In some such embodiments, the compound is:

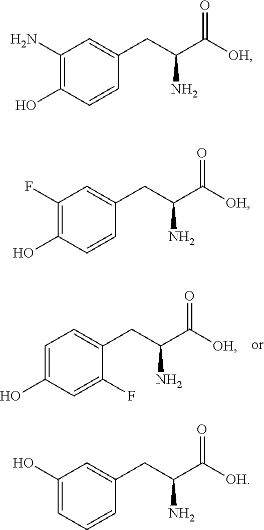 [SMILES: Nc1cc(C[C@H](N)C(=O)O)ccc1O ; N[C@@H](Cc1ccc(O)c(F)c1)C(=O)O ; N[C@@H](Cc1ccc(O)cc1F)C(=O)O ; "or" ; N[C@@H](Cc1cccc(O)c1)C(=O)O]

In other such embodiments, the compound is:

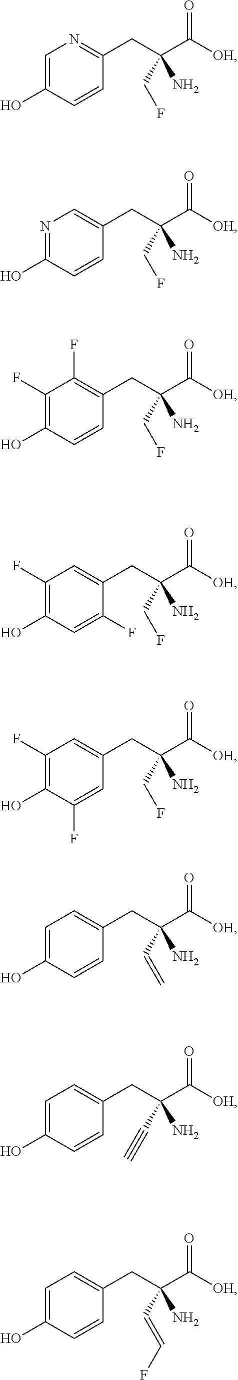

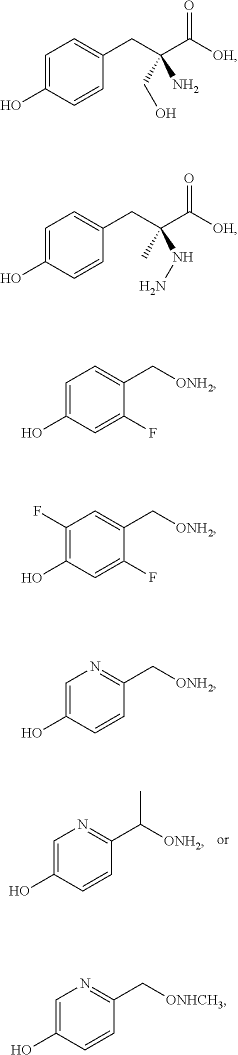

or a pharmaceutically acceptable salt thereof.

Provided herein are compounds selected from:

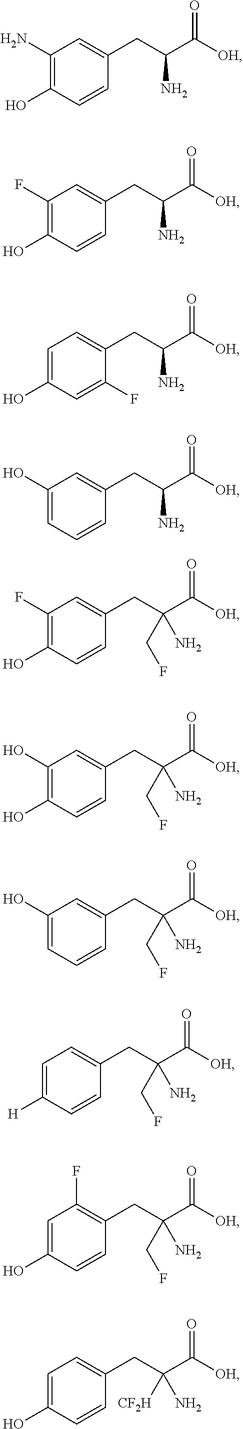

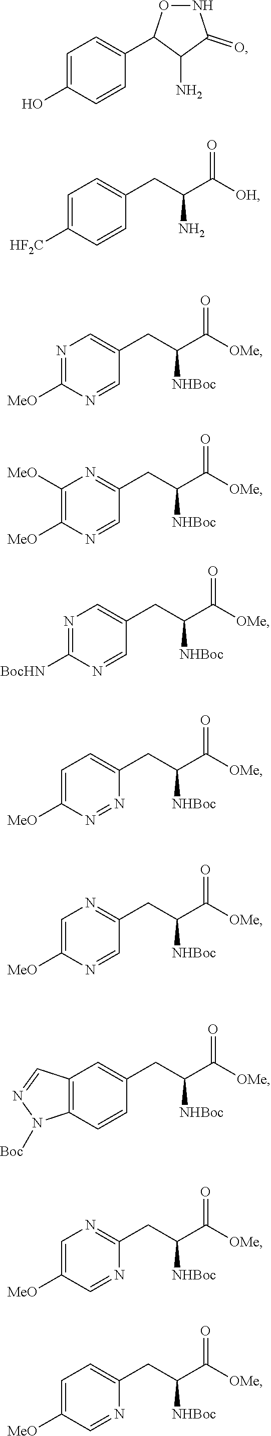

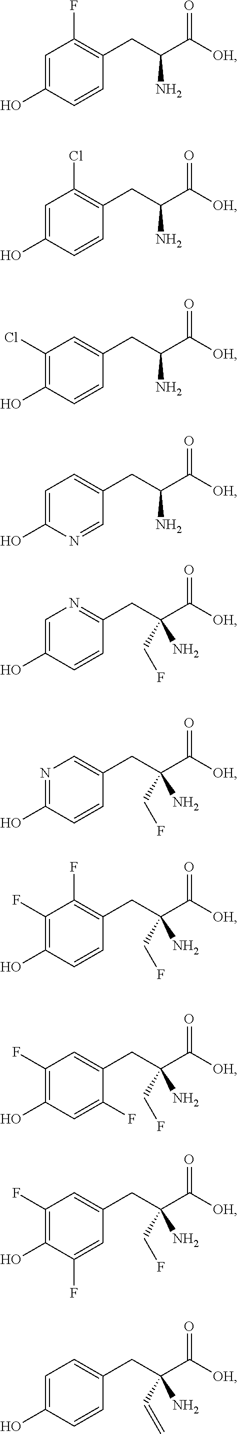

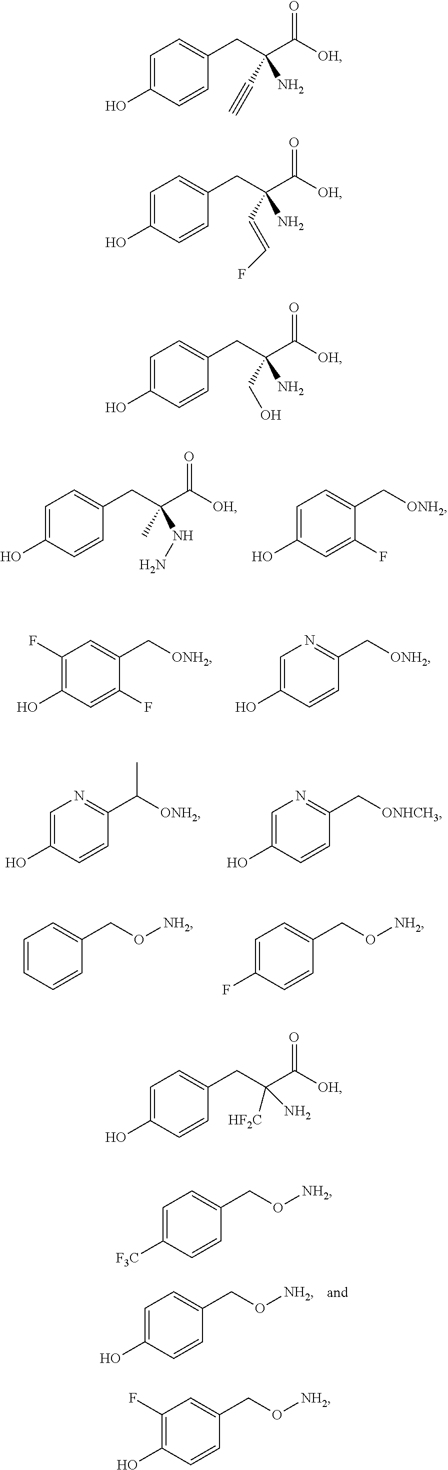

or a pharmaceutically acceptable salt thereof.

In some embodiments, the compound is

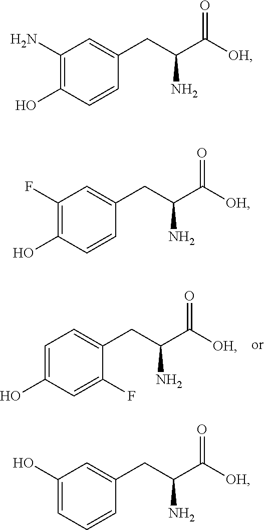 [SMILES: Nc1cc(C[C@H](N)C(=O)O)ccc1O ; N[C@@H](Cc1ccc(O)c(F)c1)C(=O)O ; N[C@@H](Cc1ccc(O)cc1F)C(=O)O ; "or" ; N[C@@H](Cc1cccc(O)c1)C(=O)O]

or a pharmaceutically acceptable salt thereof.

In some embodiments, the compound is

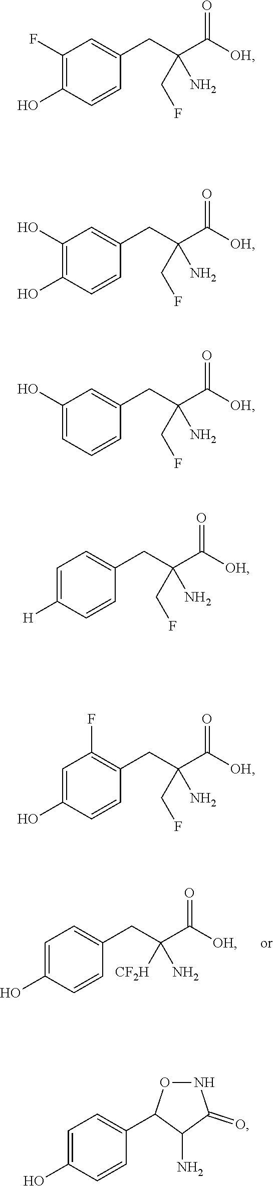

or a pharmaceutically acceptable salt thereof. In some such embodiments, the compound is

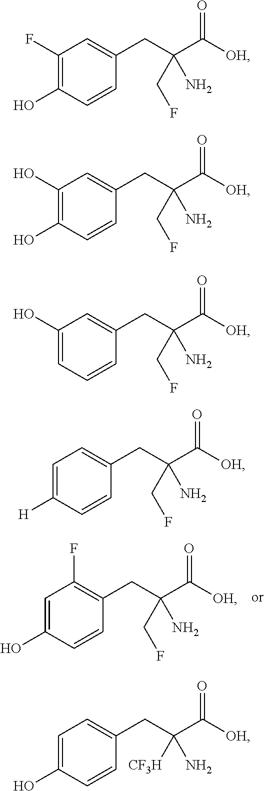

or a pharmaceutically acceptable salt thereof.

In some embodiments, the compound is

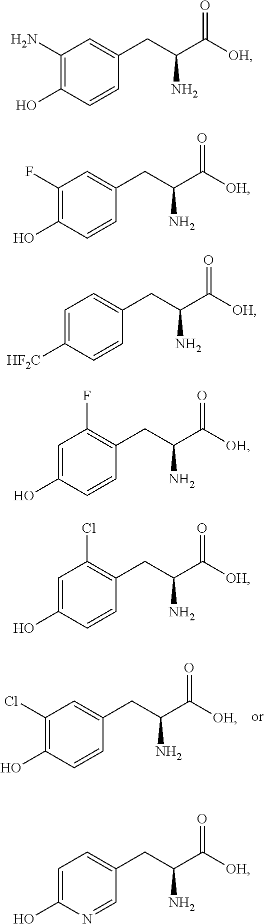

or a pharmaceutically acceptable salt thereof.

In some embodiments, the compound is

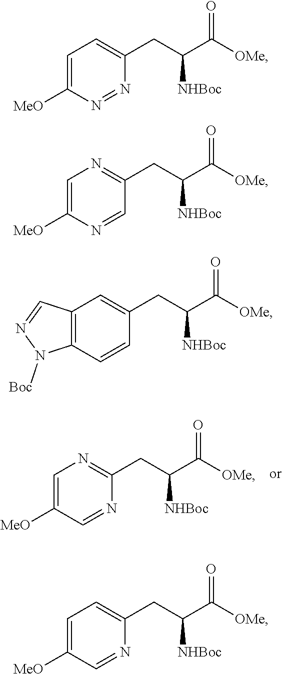 [SMILES: COC(=O)[C@H](Cc1ccc(OC)nn1)NC(=O)OC(C)(C)C ; COC(=O)[C@H](Cc1cnc(OC)cn1)NC(=O)OC(C)(C)C ; COC(=O)[C@H](Cc1ccc2c(cnn2C(=O)OC(C)(C)C)c1)NC(=O)OC(C)(C)C ; COC(=O)[C@H](Cc1ncc(OC)cn1)NC(=O)OC(C)(C)C ; "or" ; COC(=O)[C@H](Cc1ccc(OC)cn1)NC(=O)OC(C)(C)C]

or a pharmaceutically acceptable salt thereof.

In some embodiments, the compound is

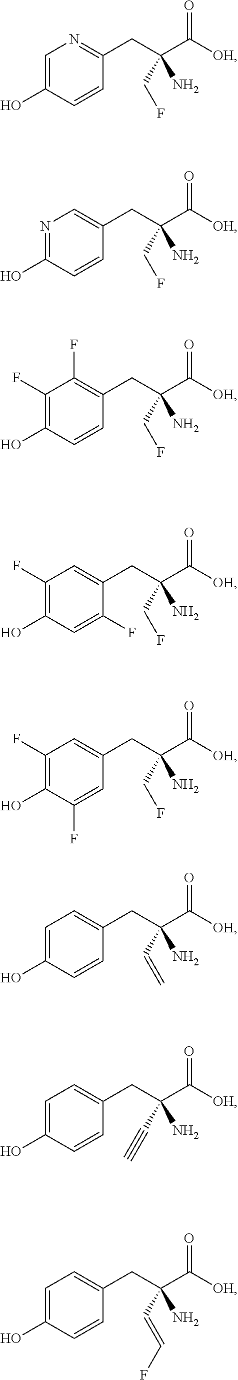

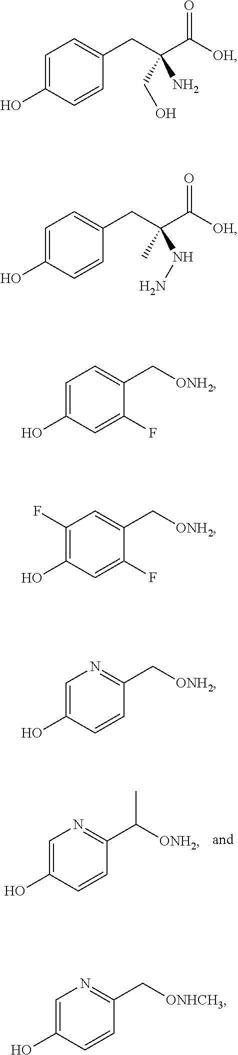

or a pharmaceutically acceptable salt thereof. In some such embodiments, the compound is not

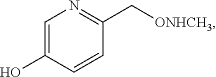

or a pharmaceutically acceptable salt thereof.

In other such embodiments, the compound is

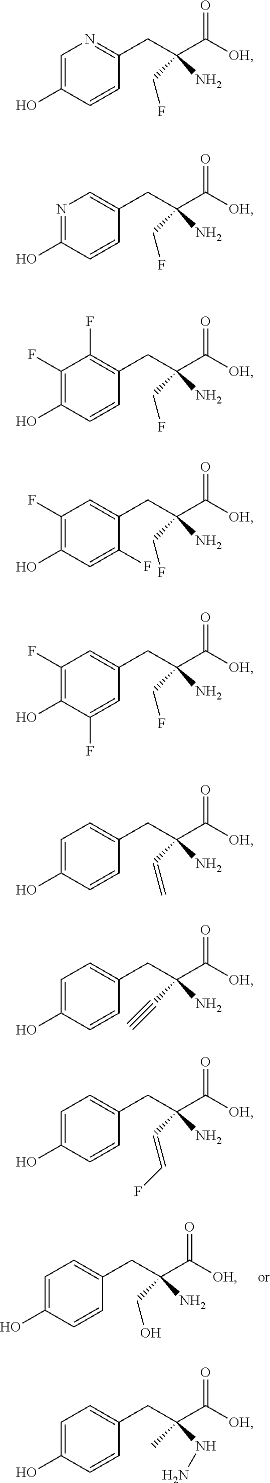

or a pharmaceutically acceptable salt thereof.

### Pharmaceutical Compositions

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. For example, some aspects of the invention relates to a pharmaceutical composition comprising a compound disclosed herein (e.g., a therapeutically effective dose of a compound disclosed herein), or a pharmaceutically acceptable salt, biologically active metabolite, solvate, hydrate, prodrug, enantiomer or stereoisomer thereof; and a pharmaceutically acceptable diluent or carrier.

As used herein, a therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl-N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di alkyl-N-(hydroxy alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Methods of Use

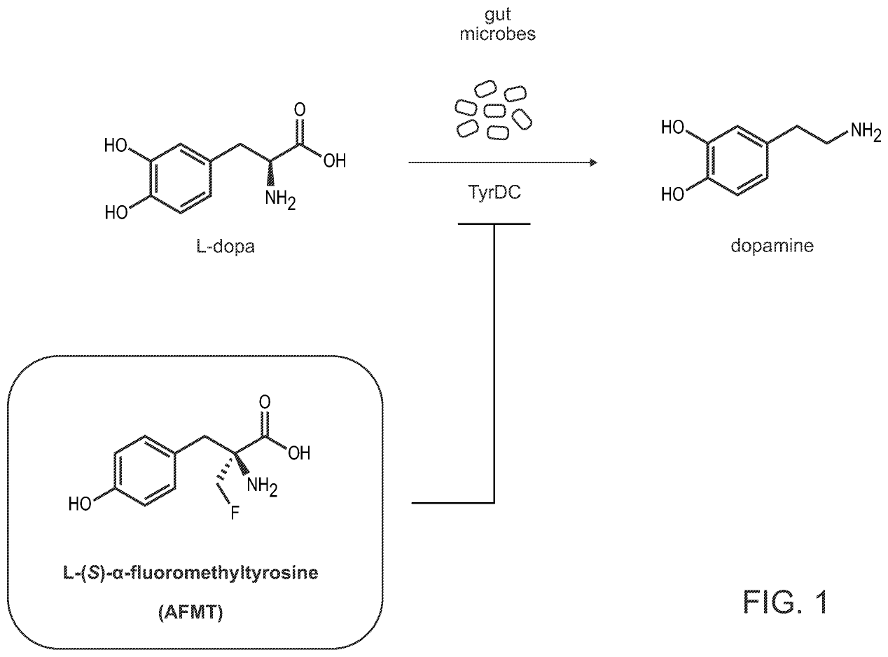

Disclosed herein are compounds and compositions for inhibiting TyrDC, which decarboxylates tyrosine, L-dopa, and related molecules. Enzyme inhibition can be measured using several methods known in the art, such as liquid chromatography-mass spectrometry. Compounds that effectively inhibit TyrDC can be identified using whole cell assays as described herein. Certain disclosed compounds can competitively inhibit TyrDC to inhibit gut microbial metabolism of L-dopa, and could thereby potentially increase bioavailability of L-dopa (FIG. 1). Such compounds may be suitable for use in treatment of neurodegenerative diseases such as Parkinson's Disease, such as by co-administration with L-dopa. In certain preferred embodiments, such compounds are characterized by IC₅₀<1 μM in whole cells and complex gut microbiota. In other preferred embodiments, such compounds are characterized by low blood brain permeability. In other preferred embodiments, such compounds are not suitable for tyrosine hydroxylation (e.g, not reactive toward tyrosine hydroxylation).

Disclosed herein are methods of treating Parkinson's Disease in a subject in need thereof, comprising administering to the subject a therapeutically effective amount of a compound of formula (I) or formula (II):

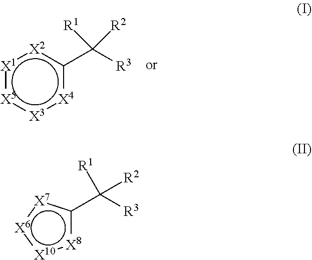

or a pharmaceutically acceptable salt thereof, wherein, as permitted by valence and stability:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or ═C(R¹¹)—;
X³ is ═N— or ═C(R′)—;
X⁴ is ═N— or ═C(R¹²)—;
provided that
   no more than two of X¹ to X⁵ are ═N—; and
   when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R′)—;
X¹⁰ is ═N—, —NR¹³—, or ═C(R⁴)—;
provided that, if at least three of X⁶ to X¹⁰ are ═N—, one of X⁶ to X¹⁰ is —NR¹³—;
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂, or

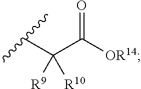

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

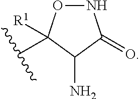

In some embodiments, R¹⁴ is, independently at each occurrence, H or CH₃. In some embodiments, R³ is

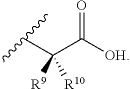

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or

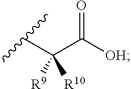

and
R¹⁰ is amino, —ONH₂, or —NHNH₂.

In some embodiments, the compound is not:

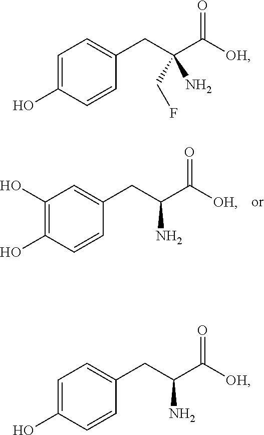

or a pharmaceutically acceptable salt of any of the foregoing. In some such embodiments, the compound is not

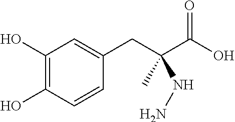 [SMILES: C[C@@](Cc1ccc(O)c(O)c1)(NN)C(=O)O]

or a pharmaceutically acceptable salt thereof.

In some such embodiments, the compound of formula (I) is:

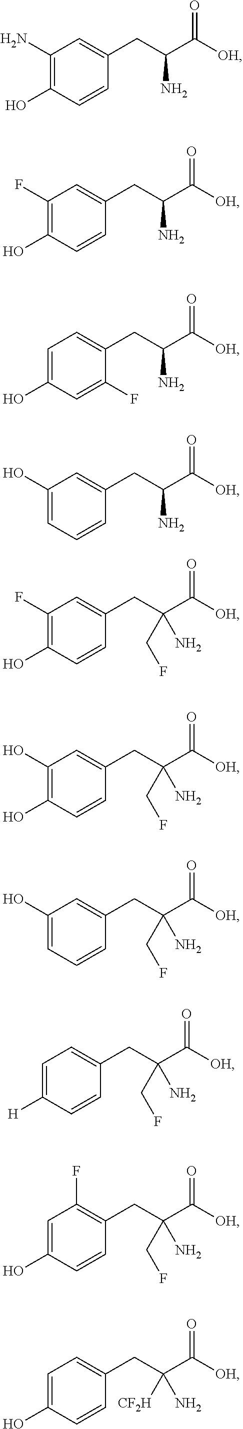

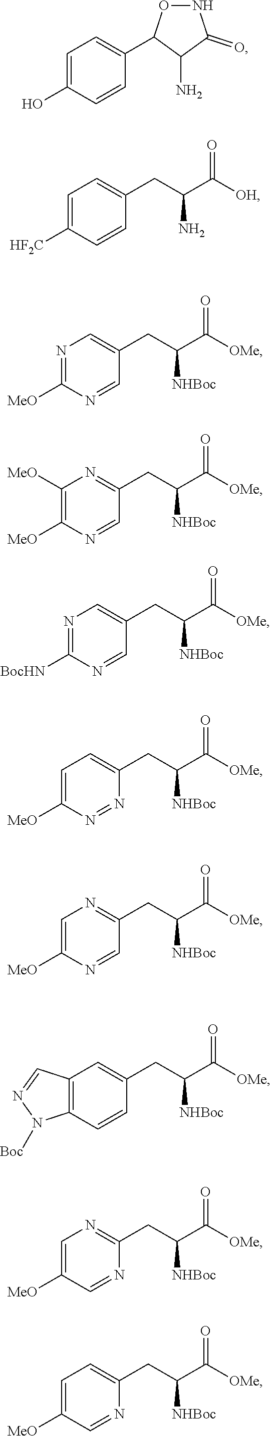

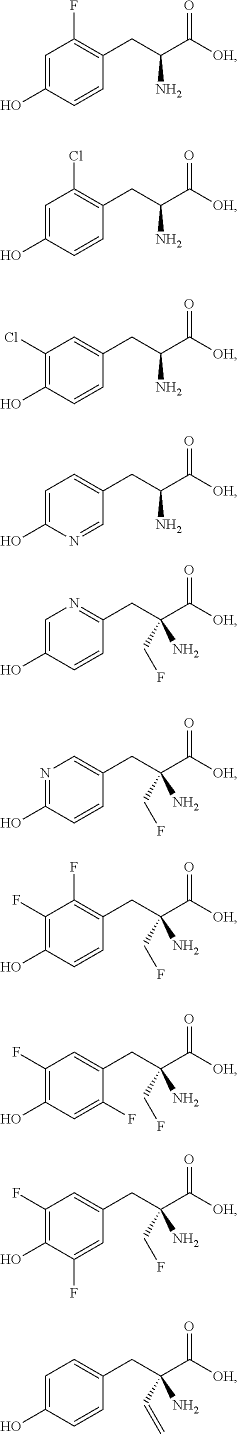

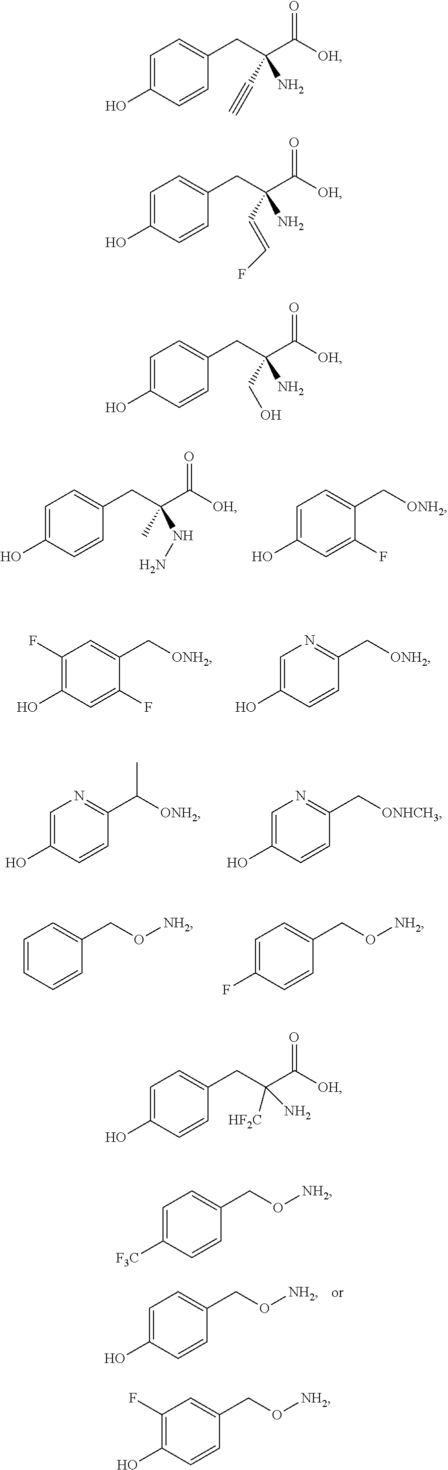

or a pharmaceutically acceptable salt thereof. In some such embodiments, the compound of formula (I) is

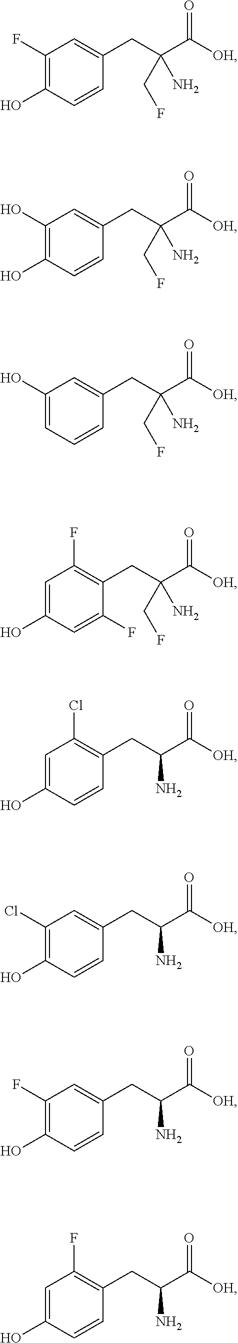

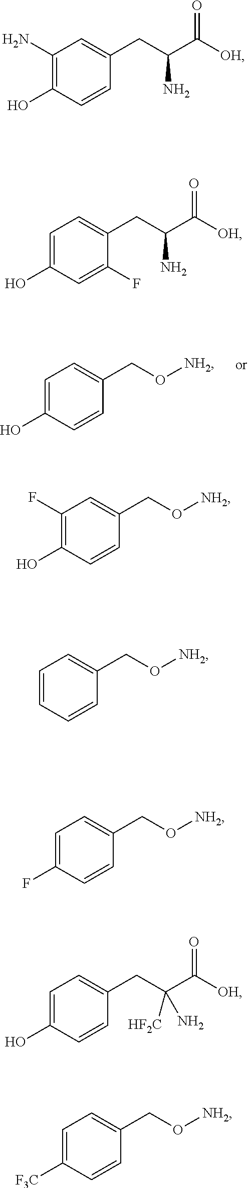

or a pharmaceutically acceptable salt thereof. In other embodiments, the compound of formula (I) is not:

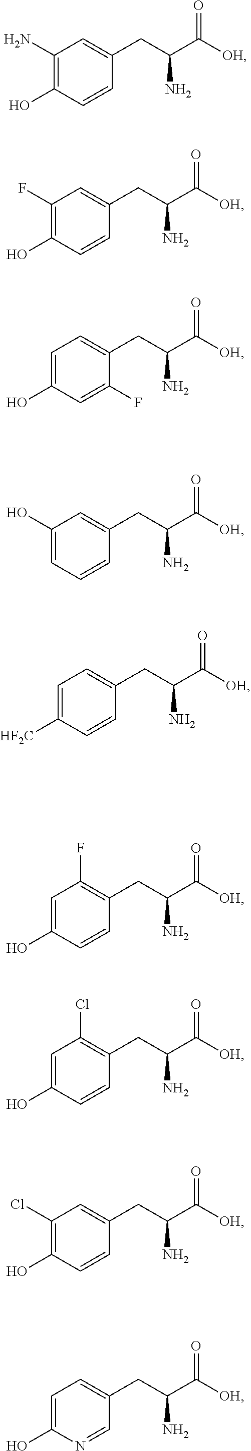

or a pharmaceutically acceptable salt thereof.

Also provided herein are methods of inhibiting a tyrosine decarboxylase (TyrDC), comprising contacting the TyrDC with a compound of formula (I) or formula (II):

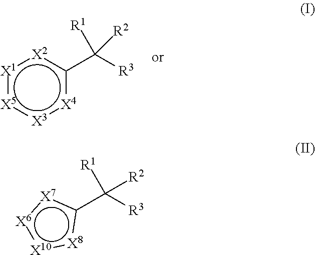

or a pharmaceutically acceptable salt thereof, wherein:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or ═C(R¹¹)—;
X³ is ═N— or ═C(R⁸)—
X⁴ is ═N— or ═C(R¹²)—; provided that
   no more than two of X¹ to X⁵ are ═N—; and
   when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R′)—;
X¹⁰ is ═N—, —NR¹³—, or ═C(R⁴)—;
provided that, if at least three of X⁶ to X¹⁰ are ═N—, one of X⁶ to X¹⁰ is —NR¹³—
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂, or

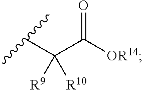

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

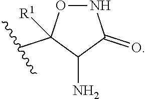

In some embodiments, R¹⁴ is, independently at each occurrence, H or CH₃. In some embodiments, R³ is

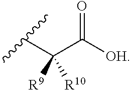

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or

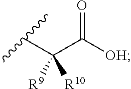

and
R¹⁰ is amino, —ONH₂, or —NHNH₂.

In some embodiments, the compound is not

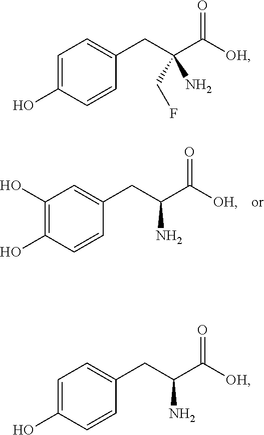

In some such embodiments, the compound is not

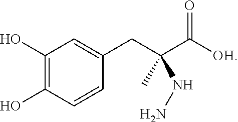 [SMILES: C[C@@](Cc1ccc(O)c(O)c1)(NN)C(=O)O]

In some embodiments, the compound of formula (I) is:

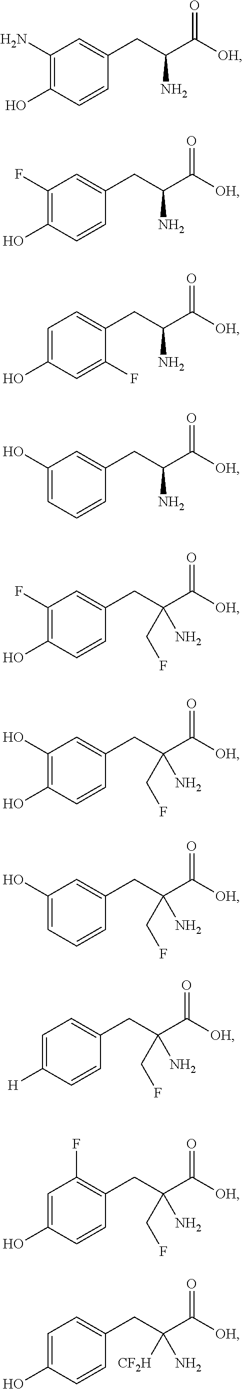

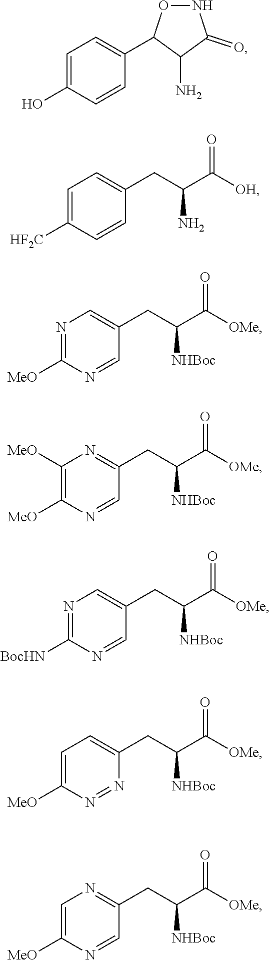

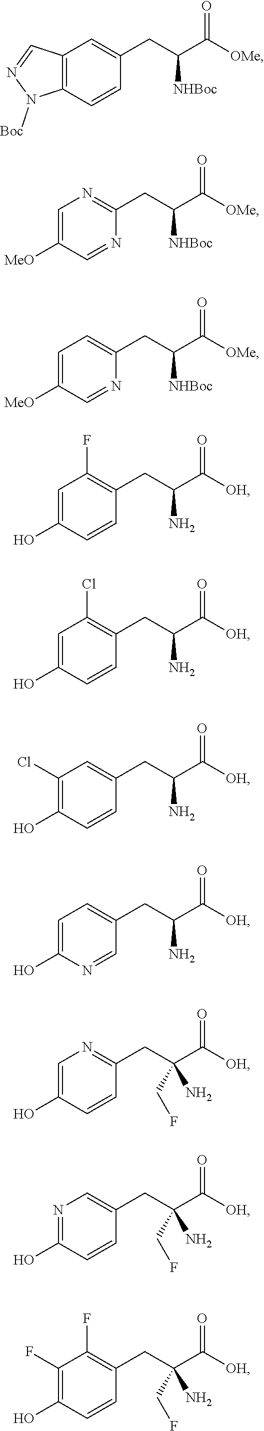

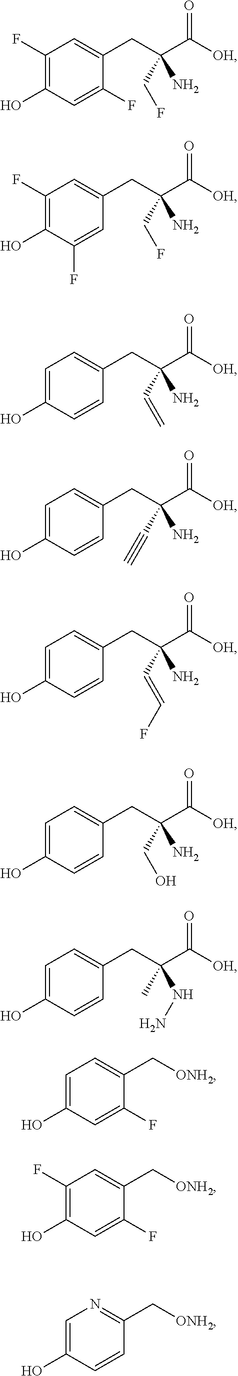

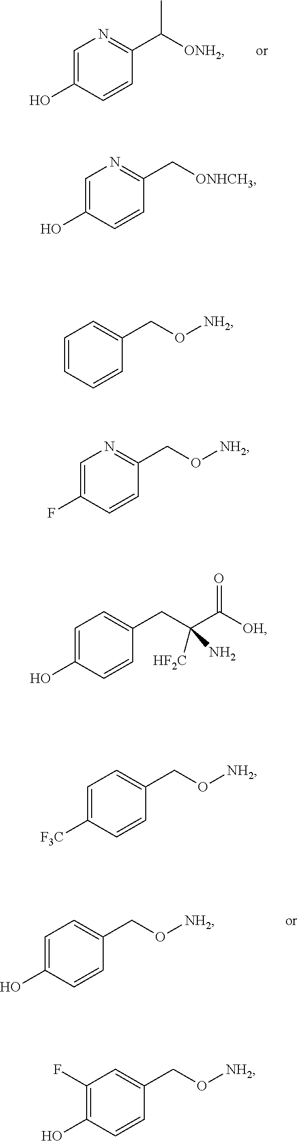

or a pharmaceutically acceptable salt thereof. In some such embodiments, the compound of formula (I) is

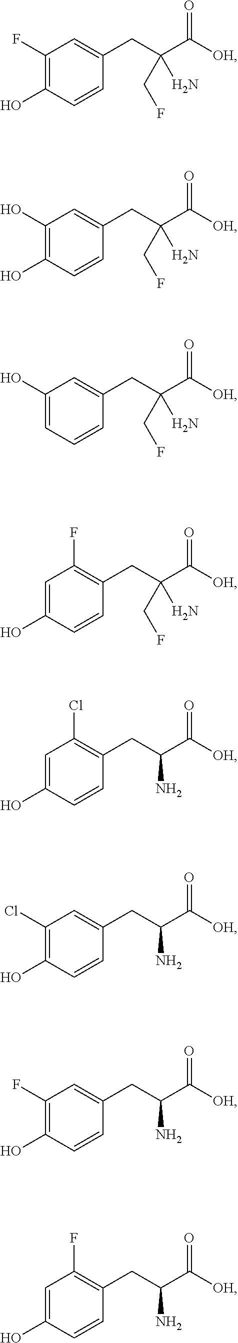

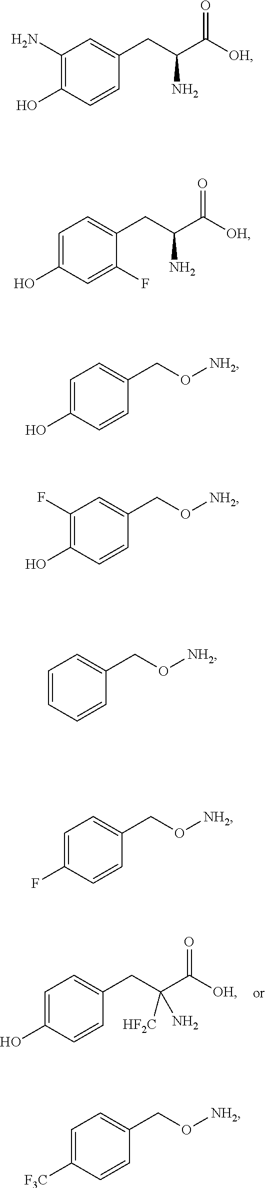

or a pharmaceutically acceptable salt thereof. In other embodiments, the compound of formula (I) is not:

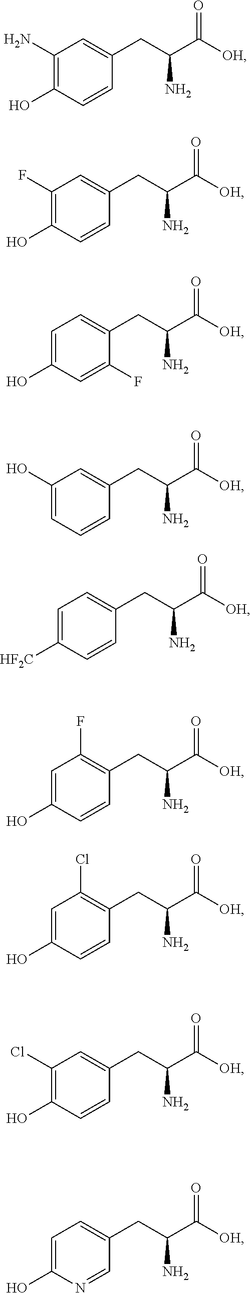

or a pharmaceutically acceptable salt thereof.

In some embodiments, the TyrDC is Enterococcus faecalis TyrDC, Enterococcus faecium TyrDC, or Providencia rettgeri TyrDC. Exemplary strains of E. faecalis include, but are not limited to, E. faecalis OG1RF, E. faecalis V1583, E. faecalis TX0104, and E. faecalis MMH594. Exemplary strains of E. faecium include, but are not limited to, E. faecium E1007, E. faecium E2134, and E. faecium TX1330.

Also provided herein are methods of treating Parkinson's Disease in a subject in need thereof, comprising ad ministering to the subject a therapeutically effective amount of a compound of formula (Ia):

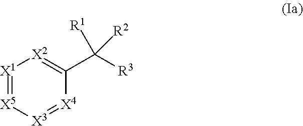

or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or

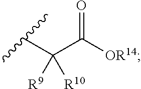

R¹⁴ is, independently at each occurrence, H or C₁₋₆ alkyl;
   R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
   R¹⁰ is amino, -NHBoc, —ONH₂, or —NHNH₂;
   or R² and R³, together with the carbon atom to which they are attached, combine to form

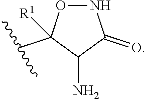

In some embodiments, R¹⁴ is, independently at each occurrence, H or CH₃. In some embodiments, wherein R³ is

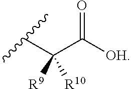

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is-NHNH₂, —ONH₂, or

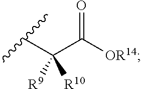

R¹⁰ is amino, —ONH₂, or —NHNH₂.

In some embodiments, the compound is not

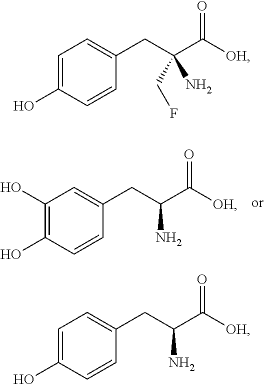

or a pharmaceutically acceptable salt thereof. In some such embodiments, the compound is not

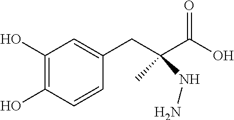 [SMILES: C[C@@](Cc1ccc(O)c(O)c1)(NN)C(=O)O]

or a pharmaceutically acceptable salt thereof.

In some embodiments, the compound of formula (Ia) is:

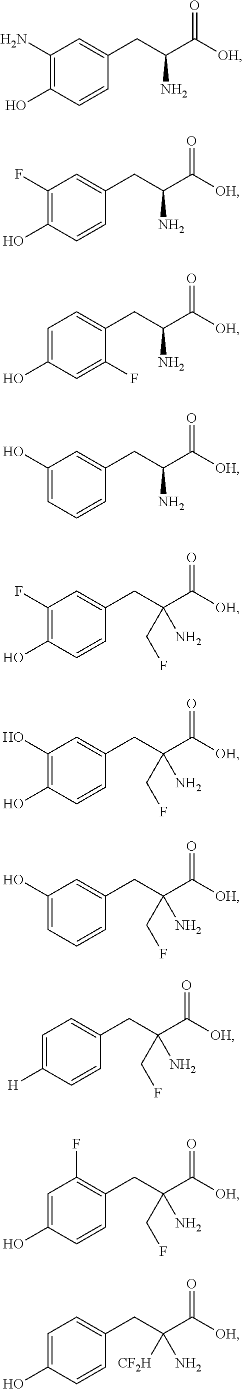

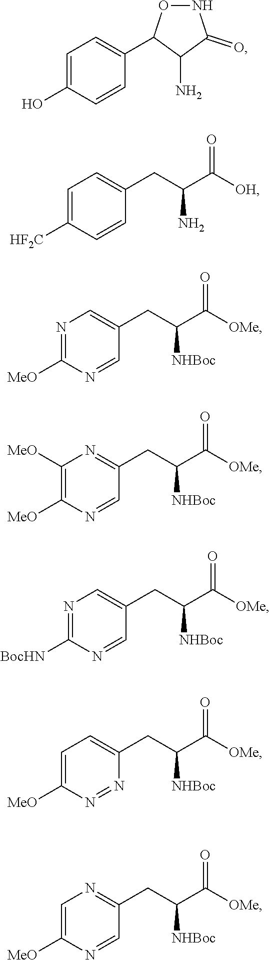

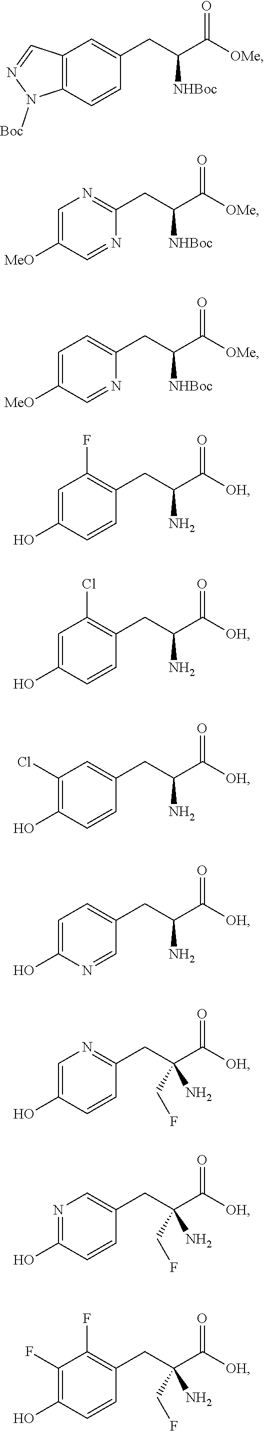

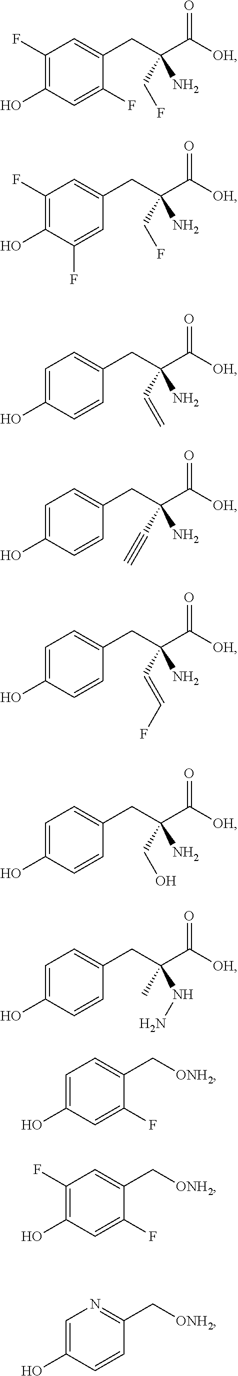

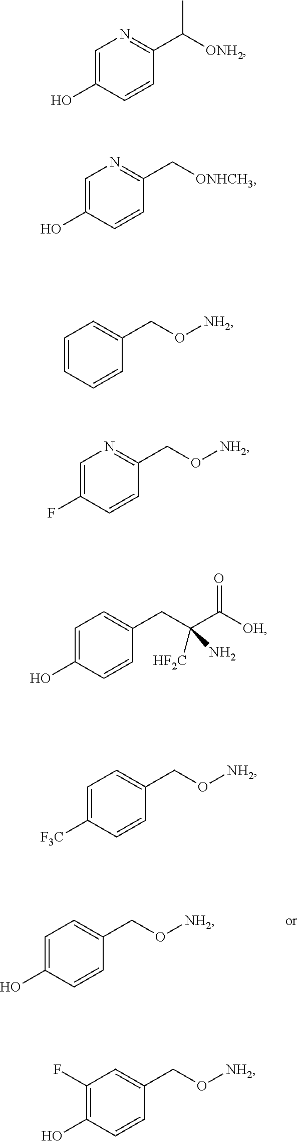

or a pharmaceutically acceptable salt thereof. In some such embodiments, the compound of formula (Ia) is

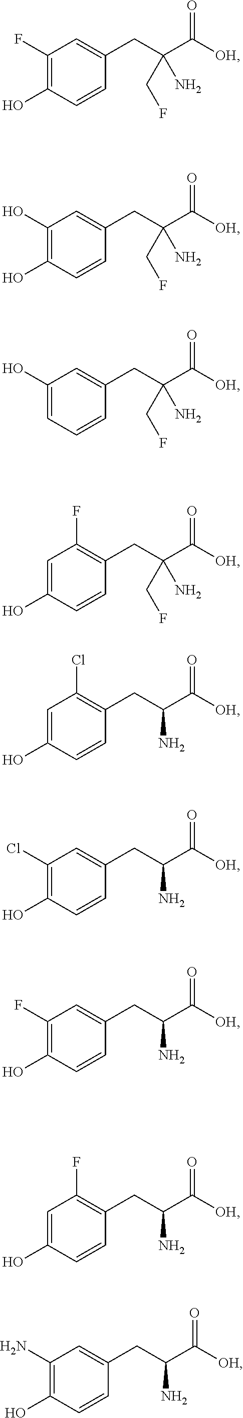

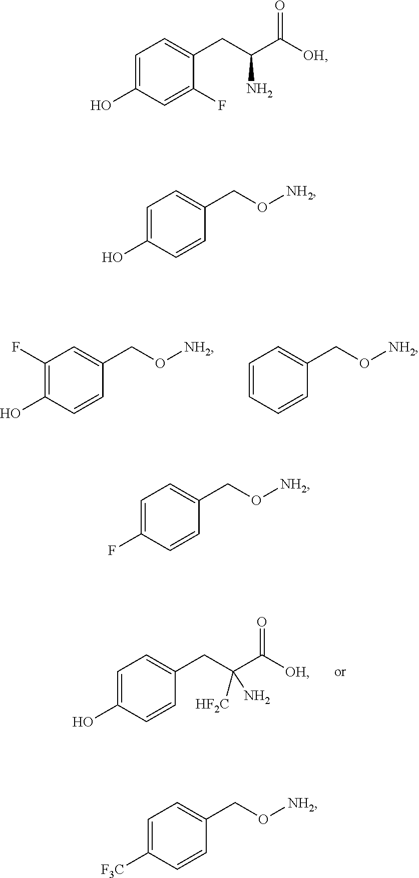

or a pharmaceutically acceptable salt thereof.

In other embodiments, the compound of formula (Ia) is not:

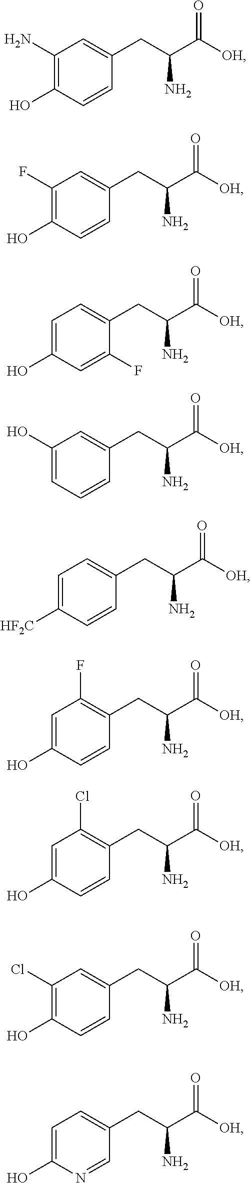

or a pharmaceutically acceptable salt thereof.

Also provided herein are methods of inhibiting a tyrosine decarboxylase (TyrDC) comprising contacting the TyrDC with a compound of formula (Ia):

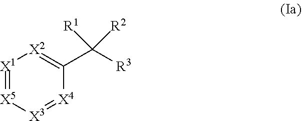

or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or

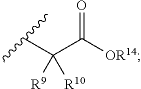

R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

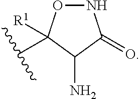

In some embodiments, R¹⁴ is, independently at each occurrence, H or CH₃. In some embodiments, R³ is

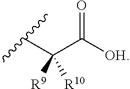

In some embodiments, R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or

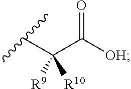

R¹⁰ is amino, —ONH₂, or —NHNH₂.

In some embodiments, the compound of formula (Ia) is not

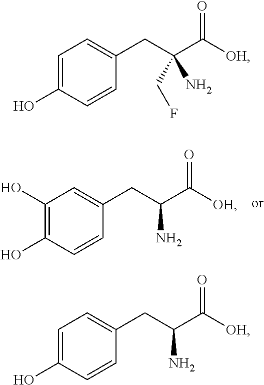

or a pharmaceutically acceptable salt thereof. In some such embodiments, the compound of formula (Ia) is not

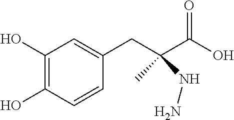 [SMILES: C[C@@](Cc1ccc(O)c(O)c1)(NN)C(=O)O]

or a pharmaceutically acceptable salt thereof.

In some embodiments, the compound of formula (Ia) is

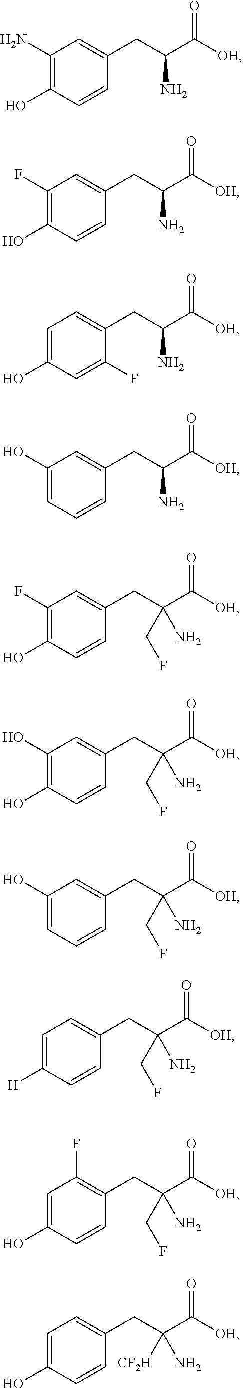

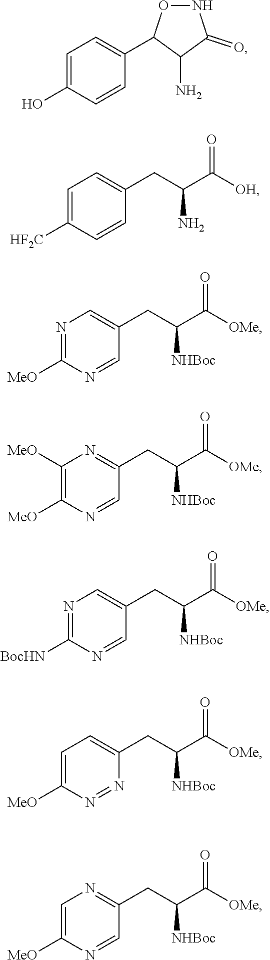

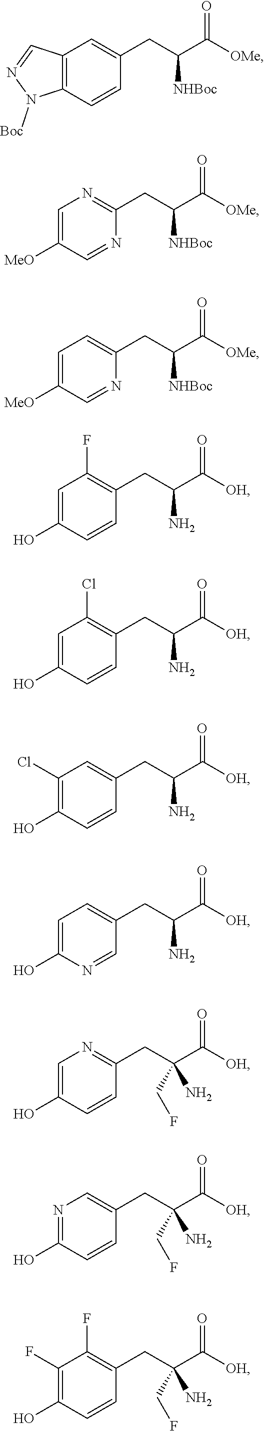

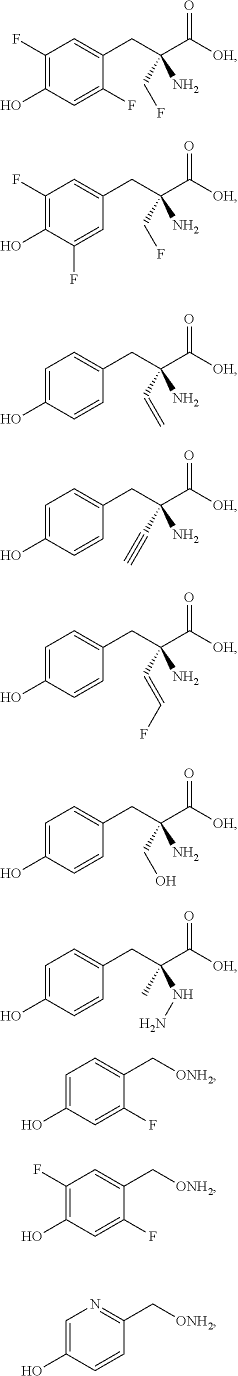

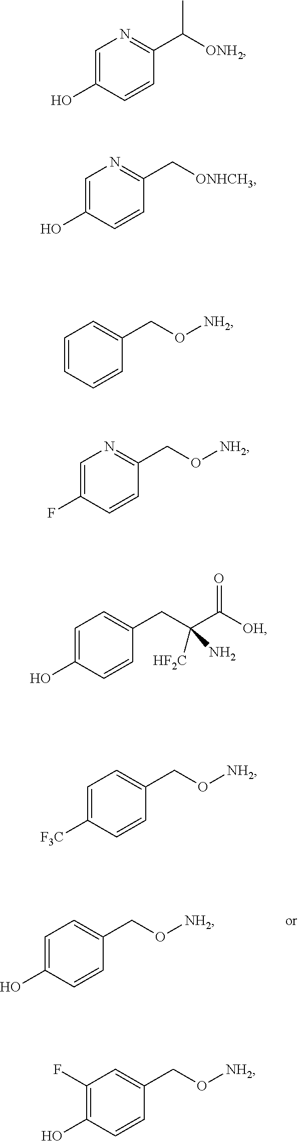

or a pharmaceutically acceptable salt thereof.

In some such embodiments, the compound of formula (Ia) is

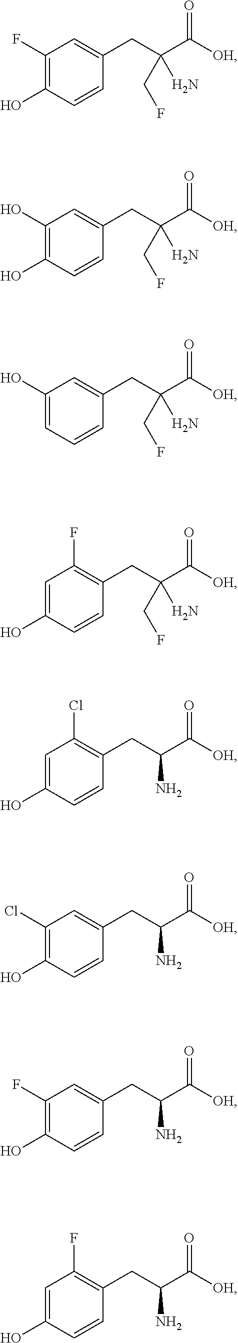

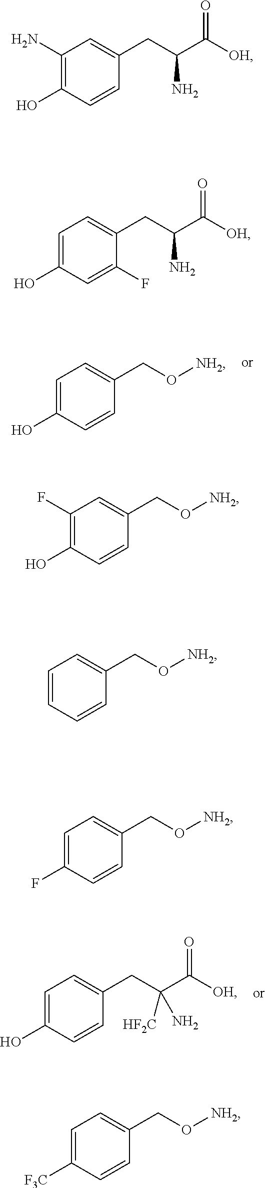

or a pharmaceutically acceptable salt thereof.

In other embodiments, the compound of formula (Ia) is not:

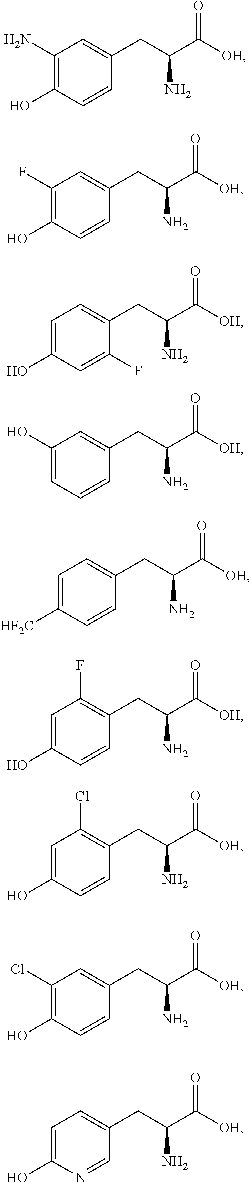

or a pharmaceutically acceptable salt thereof.

In some embodiments, the TyrDC is Enterococcus faecalis TyrDC, Enterococcus faecium TyrDC, or Providencia rettgeri TyrDC. Exemplary strains of E. faecalis include, but are not limited to, E. faecalis OG1RF, E. faecalis V1583, E. faecalis TX0104, and E. faecalis MMH594. Exemplary strains of E. faecium include, but are not limited to, E. faecium E1007, E. faecium E2134, and E. faecium TX1330.

### EXEMPLIFICATION

The invention now being generally described, it will be more readily understood by reference to the following examples, which are included merely for purposes of illustration of certain aspects and embodiments of the present invention, and are not intended to limit the invention.

### Scaffold Design

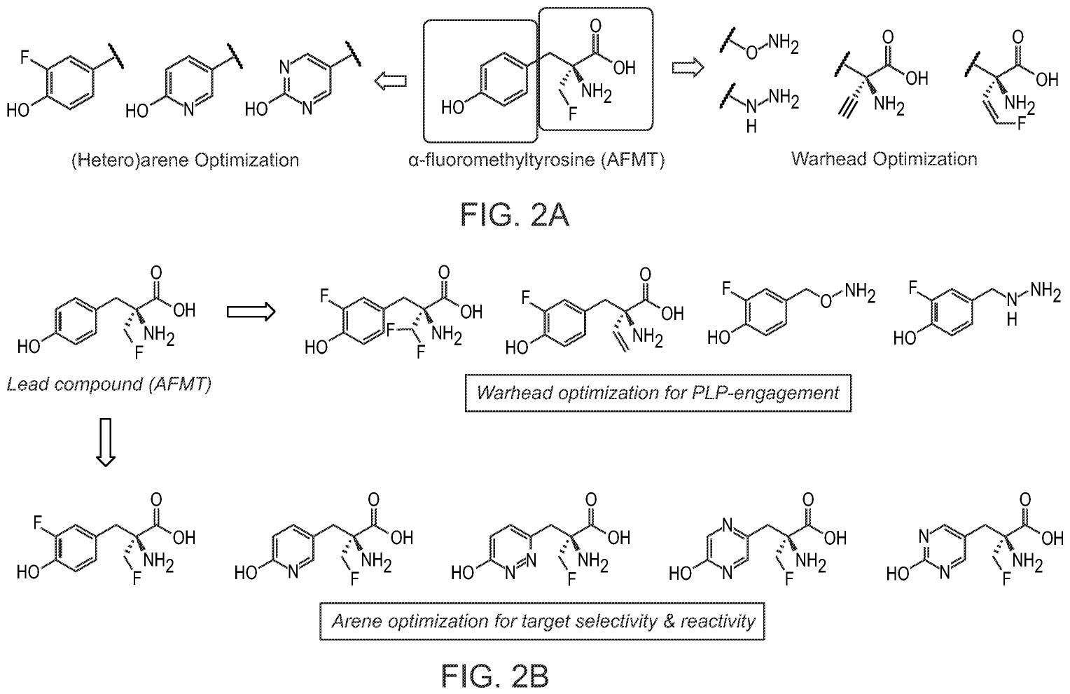

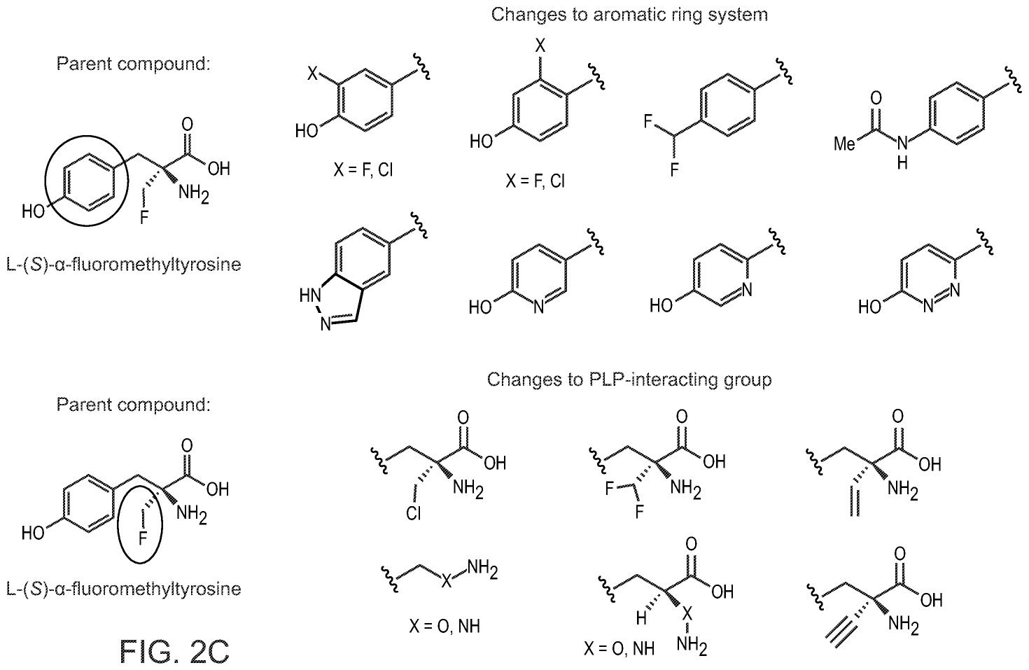

Employing small molecules to inhibit the activity of microbial TyrDC toward L-dopa has the potential to improve L-dopa therapy for Parkinson's disease patients. The discovery of α-fluoromethyltyrosine (AFMT) as an inhibitor of L-dopa decarboxylation by the TyrDC-expressing bacterium E. faecalis led us to seek novel inhibitors with improved potency and selectivity. We have investigated: (1) the aromatic scaffold and (2) the nucleophilic warhead that engages the pyridoxal phosphate (PLP) cofactor of TyrDC (FIGS. 2A-2C).

### Identification of Aromatic Scaffolds for TyrDC Inhibitor

TyrDC exhibits a preference for substrates that resemble tyrosine. Indeed, tyrosine or L-dopa undergoes decarboxylation in whole cells at a faster rate in comparison to m-tyrosine. AFMT was shown to be a potent TyrDC inhibitor in whole cells (EC₅₀=1.4 μM). Thus, this project set out to more fully assess the potential for further substitution of the aromatic ring.

The current synthesis of AFMT is linear, lengthy, and limited in scope. We have developed a divergent synthetic strategy for the construction of AFMT analogs (vide infra). To more rapidly assess tolerance of the enzyme (and cellular transporters) to different substituents on the aromatic ring of tyrosine, we chose to employ the native decarboxylation activity of TyrDC as a structural probe to survey a wide range of tyrosine analogs. Tyrosine analogs are readily available via commercial and synthetic sources, which renders a high throughput approach feasible.

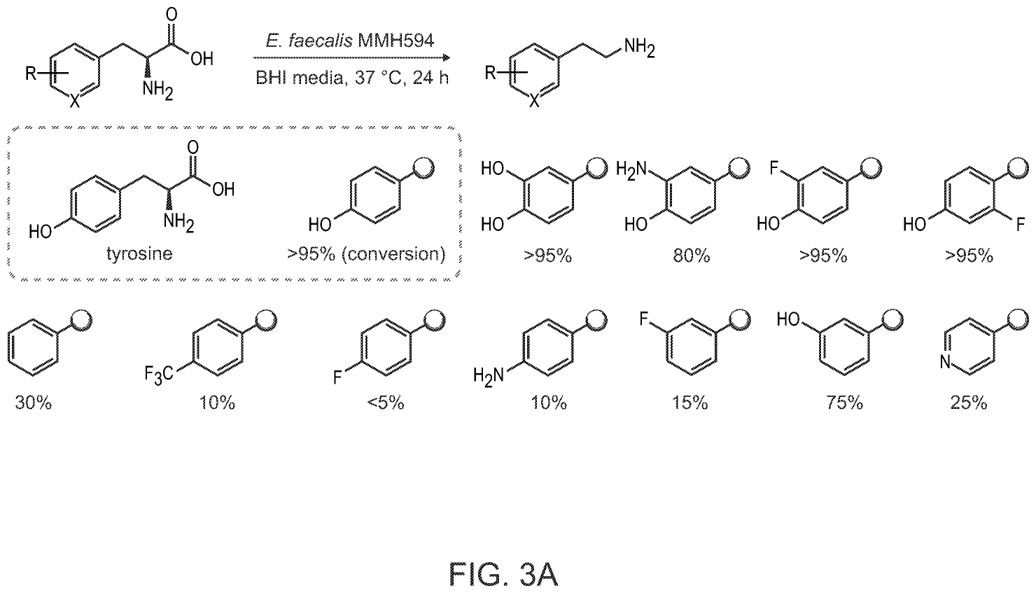

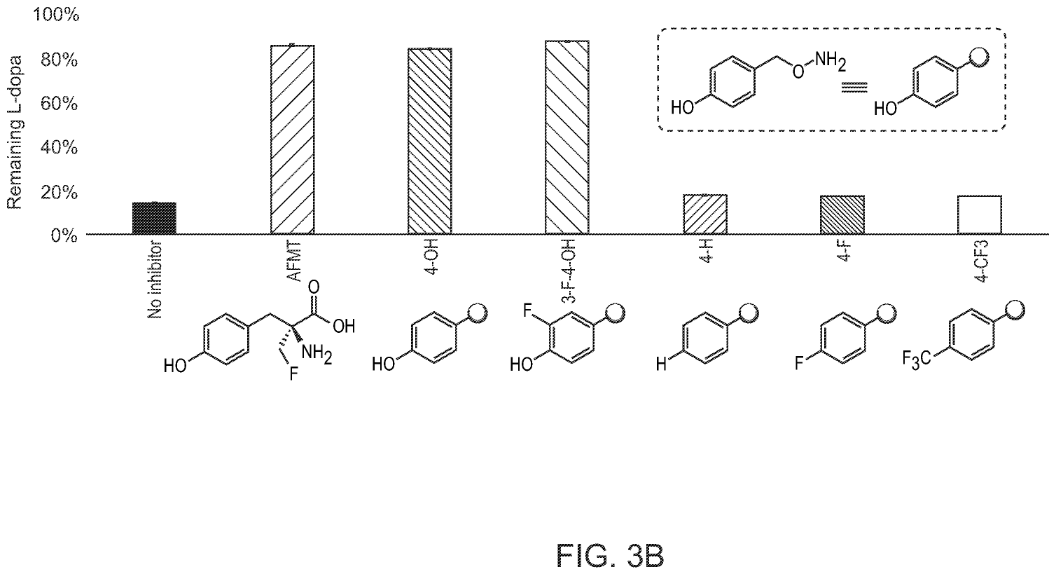

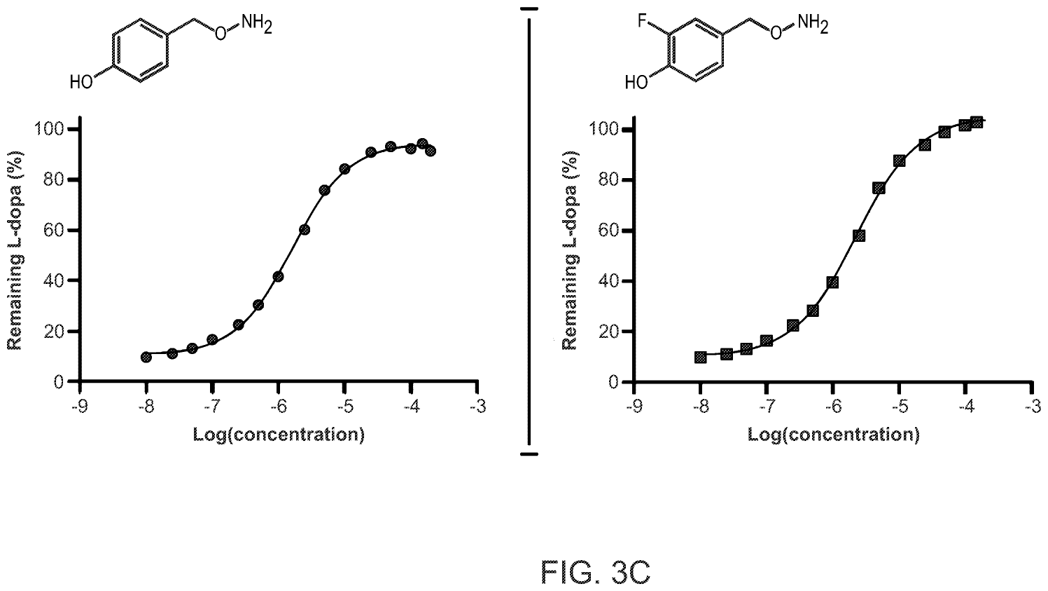

Thus, a library of commercially available tyrosine and phenylalanine derivatives were evaluated for decarboxylation by Enterococcus faecalis MMH594 whole cells; decarboxylation efficiency of a given tyrosine analog was used to identify favorable aromatic fragments, with high conversion indicating a favorable arene scaffold. We observed that decarboxylation was most effective when a hydroxyl group was present at the para position, and that substitutions at other positions on the arene were well tolerated as long as this para-hydroxyl group was present (FIG. 3A). To quickly assess whether the trend in native decarboxylation activity can be translated to inhibitory activity, we evaluated benzyl aminooxy compounds with similar aromatic elements as inhibitors for L-dopa decarboxylation in E. faecalis MMH594 whole cells (10 μM inhibitor); high L-dopa recovery indicated high inhibitory efficiency. Studies have demonstrated benzyl aminooxy compounds are inhibitors for PLP-dependent decarboxylase enzymes. In addition, these compounds are either commercially available or can be accessed in a two-step synthetic sequence (European Journal of Medicinal Chemistry 2016, 108, 564-576). As shown, inhibitors without a para-hydroxyphenyl motif are not effective towards L-dopa decarboxylation in whole cells (FIG. 3B). Aminooxy compounds with this structural motif can inhibit L-dopa decarboxylation in E. faecalis cultures at low concentrations. Notably, the potency of 4-hydroxybenzyl aminooxy and 3-fluoro-4-hydroxybenzyl aminooxy compounds were similar to AFMT, with EC₅₀ values of 1.5 μM and 2.2 μM, respectively (FIG. 3C).

### Divergent Synthesis of Tyrosine Analogs by Photocatalysis

Tyrosine analogs were prepared by applying the conditions provided in JACS 2016, 138, 8084-8087, which couples alkyl bromides with aryl or heteroaryl bromides. The alkyl bromide can be prepared directly in decagram scale from a protected serine. While unprotected heterobromoarenes such as

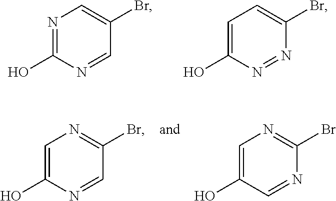 [SMILES: Oc1ncc(Br)cn1 ; Oc1ccc(Br)nn1 ; Oc1cnc(Br)cn1 ; "and" ; Oc1cnc(Br)nc1]

did not afford the desired product, methyl-protected substrates such as

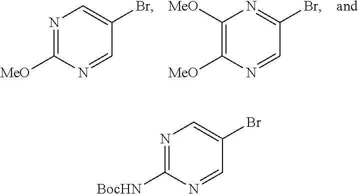 [SMILES: COc1ncc(Br)cn1 ; COc1ncc(Br)nc1OC ; "and" ; CC(C)(C)OC(=O)Nc1ncc(Br)cn1]

yielded the desired coupling products. Additional heterobromoarenes to be tested in the coupling reaction include

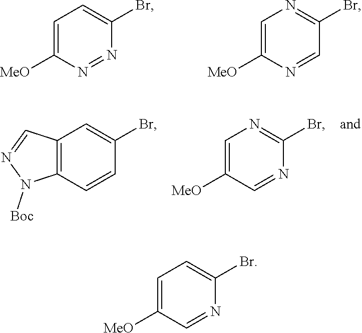 [SMILES: COc1ccc(Br)nn1 ; COc1cnc(Br)cn1 ; CC(C)(C)OC(=O)n1ncc2cc(Br)ccc21 ; COc1cnc(Br)nc1 ; "and" ; COc1ccc(Br)nc1]

### Tyrosine Analog Decarboxyation Activity

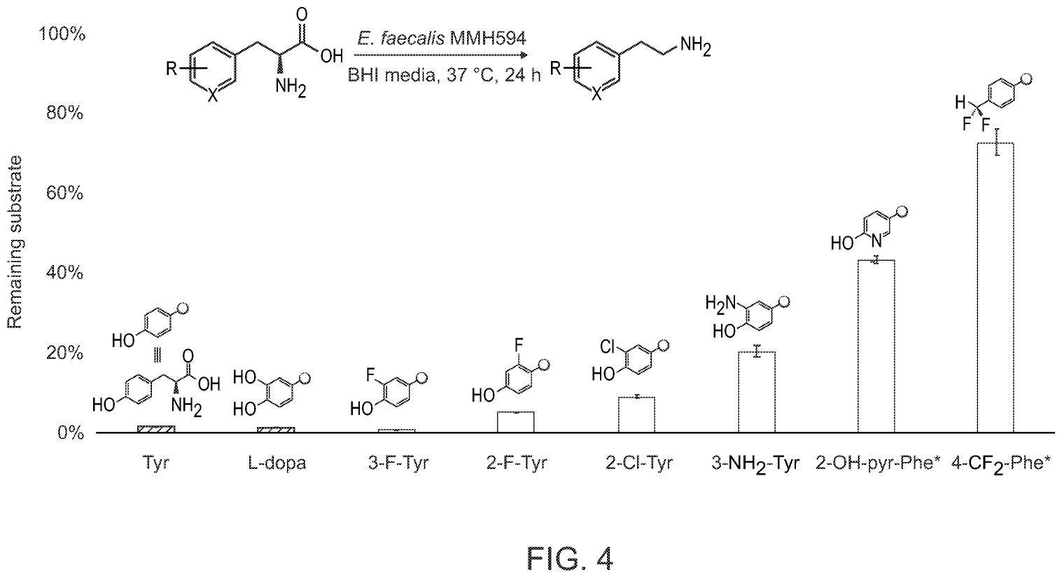

We also examined the efficiency with which E. faecalis MNM594 decarboxylates these new unnatural amino acids (FIG. 4). Substrate (500 μM) was incubated with E. faecalis WT MMH594 in BHI media under anaerobic conditions at 37° C. for 18-24 hours (done in triplicate). The amount of remaining starting material was analyzed by LC-MS-MRM. Results were normalized to samples incubated with E. faecalis AtyrDC3. We observed that substitution of the p-hydroxyl substituent of L-Tyr with a difluoromethyl group limits the decarboxylation efficiency, suggesting that inhibitors with this bioisosteric substituent would not be as effective as those bearing native 4-hydroxyphenyl group. In addition, the 2-hydroxypyridyl analog was tolerated, being converted to the corresponding amine to to tyrosine, in low efficiency.

### AFMT Analog Synthesis

Synthesis of α-fluoromethyl amino acids has typically been a laborious process. We have investigated different strategies to establish a shorter and more robust route to synthesize AFMT analogs. Recently, we found a relatively quick process that converts the amino acid to its corresponding α-fluoromethyl analog. As shown below, p-methoxyphenylalanine (8) was converted to the oxazolidinone 9 to facilitate the hydroxymethyl motif installation. The key deoxyfluorination step was examined under different reported protocols. AlkylFluor was effective in giving the desired product 12 in acceptable yield. Deprotection of alkyl fluoride 12 should yield the corresponding α-fluoromethylamino acids. Protocols for deprotection of alkyl fluorides such as 12 are well established. Overall, the sequence comprises robust transformations that do not require specialized reaction apparatus.

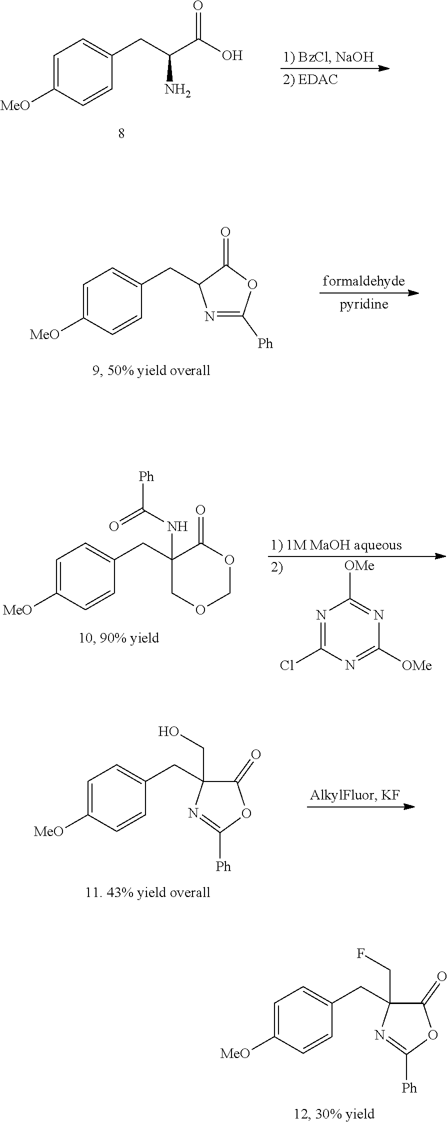 [SMILES: COc1ccc(C[C@H](N)C(=O)O)cc1 ; COc1ccc(CC2N=C(c3ccccc3)OC2=O)cc1 ; COc1ccc(CC2(NC(=O)c3ccccc3)COCOC2=O)cc1 ; COc1nc(Cl)nc(OC)n1 ; COc1ccc(CC2(CO)N=C(c3ccccc3)OC2=O)cc1 ; COc1ccc(CC2(CF)N=C(c3ccccc3)OC2=O)cc1]

### Inhibitory Effect of AFMT Analogs

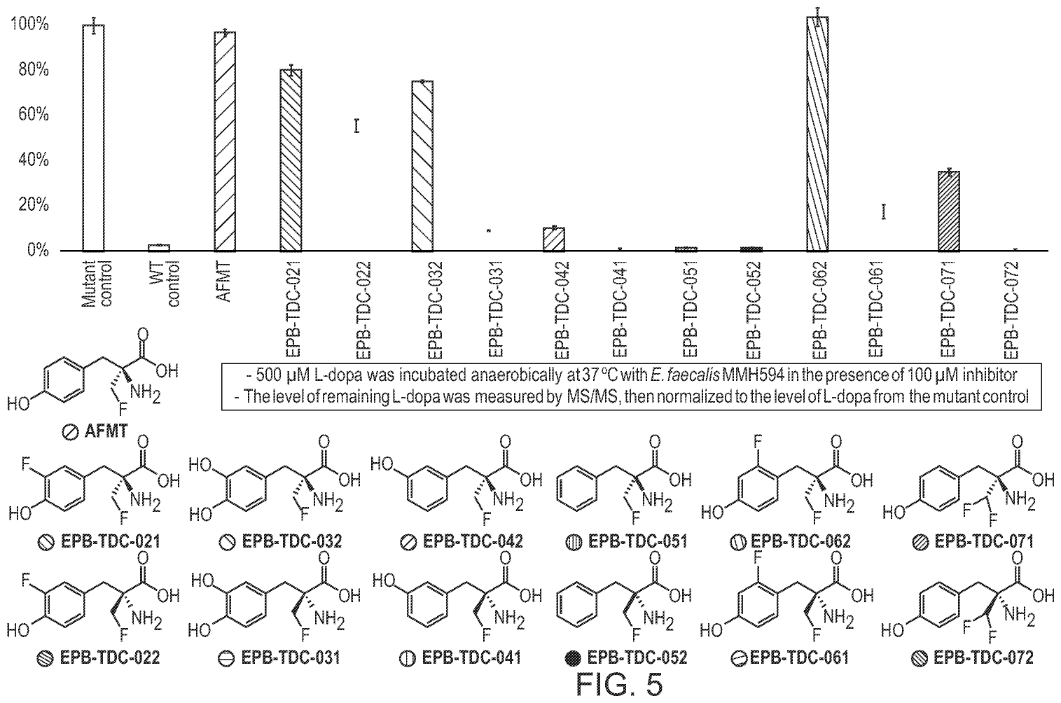

AFMT analogs were evaluated for their inhibitory effects towards L-dopa decarboxylation using the same protocol as described above. 500 μM L-dopa was incubated anaerobically at 37° C. with E. faecalis MMH594 in the presence of 100 μM inhibitor for 18-24 h. The level of remaining L-dopa was measured by MS/MS and then normalized to the level of L-dopa from a mutant control. As shown in FIG. 5, EPB-TDC-021, -031, -041, and -062 were found to retain >80% of L-dopa. Since (S)-AFMT is the active AFMT enantiomer, the stereochemical assignments in FIG. 5 assume that the enantiomer with the higher inhibitory activity carries the (5) configuration. Chiral configuration will be unambiguously determined via crystallography in the future.

Fluoride substituents are well-tolerated on the aromatic motif, as shown by EPB-TDC-021 and -062. In addition, the activity of EPB-TDC-41 suggests that a meta-hydroxyl group can be similarly effective to its para-counterpart, and comparing the activity of AFMT, EPB-TDC-031, and -041 to -051/-052 suggests the importance of a hydroxyl group for inhibitory activity.

These results also show the importance of the nucleophilic warhead in inhibitor design. In our original study, carbidopa was found to be ineffective in limiting L-dopa decarboxylation by E. faecalis. Comparing carbidopa with EPB-TDC-031 shows that the α-fluoromethyl warhead in 031 is more effective than the α-methyl-hydrazine of carbidopa. Furthermore, the comparison between AFMT and EPB-TDC-071 revealed that the α-fluoromethyl moiety is more effective in comparison to the α-difluoromethyl warhead.

### Assessing Inhibitor Effects on L-Dopa Bioavailability

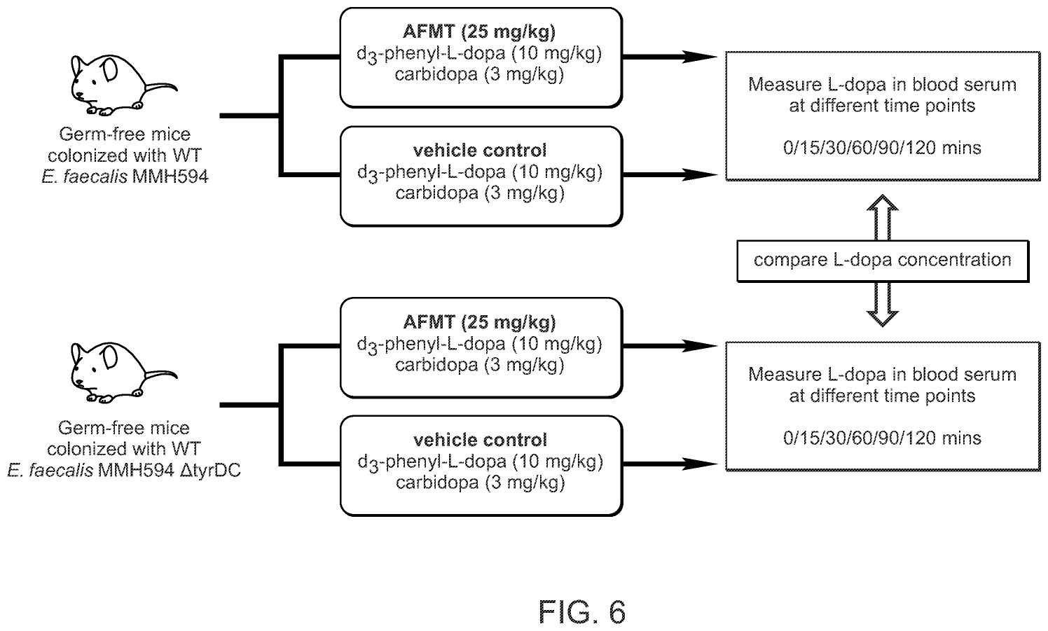

Preliminary mouse experiments to examine the efficacy of AFMT in increasing serum L-dopa levels support a correlation between this effect and TyrDC inhibition. As shown in FIG. 6, gnotobiotic mice will be colonized with either E. faecalis MMH594 WT or E. faecalist MMH594 mutant wherein tyrDC is interrupted by a Tet-cassette. For each group, a cocktail of AFMT/L-dopa/carbidopa or the corresponding vehicle control will be administered. The L-dopa concentration in the serum will be monitored periodically.

A comparison of serum L-dopa concentrations in the WT group will provide supporting evidence for the effect of AFMT on the bioavailability of L-dopa. Next, a comparison between the WT and mutant groups will provide an indication that the increase in L-dopa levels is due to inhibiting E. faecalis L-dopa metabolism by TyrDC. The results of this initial experiment may inform follow up experiments to optimize the dosing of AFMT and will also inform experiments on subsequent generations of inhibitors. After efficacy and safety of AFMT and other inhibitors has been assessed, studies will begin with an animal model of Parkinson's disease. Specifically, changes in serum and brain L-dopa levels will be assessed in parallel to motor function in the animals.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula (I) or formula (II): 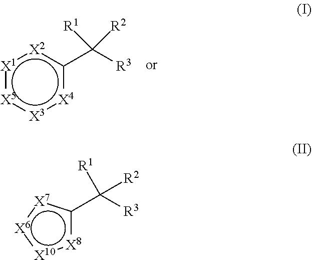
or a pharmaceutically acceptable salt thereof, wherein, as permitted by valence and stability:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or —C(R¹¹)═;
X³ is —N═ or —C(R⁸)—;
X⁴ is ═N— or ═C(R¹²)—;
provided that
no more than two of X¹ to X⁵ are ═N—; and
when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R⁸)—;
X¹⁰ is ═N—, —NR¹³—, or ═C(R⁴)—;
provided that, if at least three of X⁶ to X¹⁰ are ═N—, one of X⁶ to X¹⁰ is —NR¹³—;
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂ or 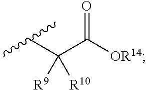
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

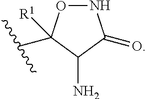

2. The compound of claim 1, wherein R¹⁴ is, independently at each occurrence, H or CH₃.

3. The compound of claim 1, wherein R³ is

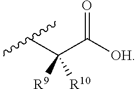

4. The compound of claim 1, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or 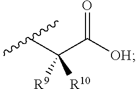
and
R¹⁰ is amino, —ONH₂, or —NHNH₂.

5. The compound of any one of claims 1-4 provided the compound is not

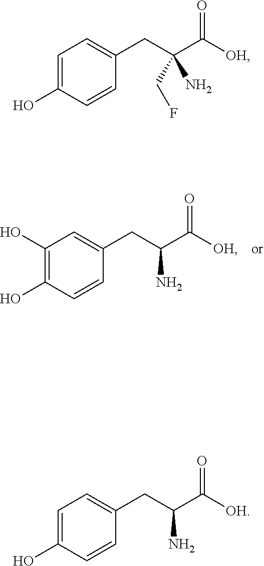 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

6. The compound of claim 5, further provided that the compound is not

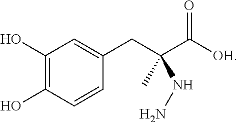 [SMILES: C[C@@](Cc1ccc(O)c(O)c1)(NN)C(=O)O]

7. The compound of any one of the preceding claims, wherein R³ is

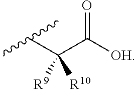

8. The compound of any one of the preceding claims, wherein X¹ is CR⁷, X² is CR¹¹, X³ is CR⁸, X⁴ is CR¹², and X⁵ is CR⁴; or X⁶ is CR⁷, X⁷ is CR¹¹, X⁸ is CR⁸, and X¹⁰ is CR⁴.

9. The compound of claim 8, wherein R⁷ is H and R⁸ is H.

10. The compound of any one of the preceding claims, wherein X⁵ is CR⁴ and R⁴ is hydroxyl; or X¹⁰ is CR⁴ and R⁴ is hydroxyl.

11. The compound of any one of the preceding claims, wherein R¹ is H and R² is H.

12. The compound of any one of the preceding claims, wherein R⁷ is hydroxyl, halogen, or amino.

13. The compound of any one of the preceding claims, wherein the compound is: 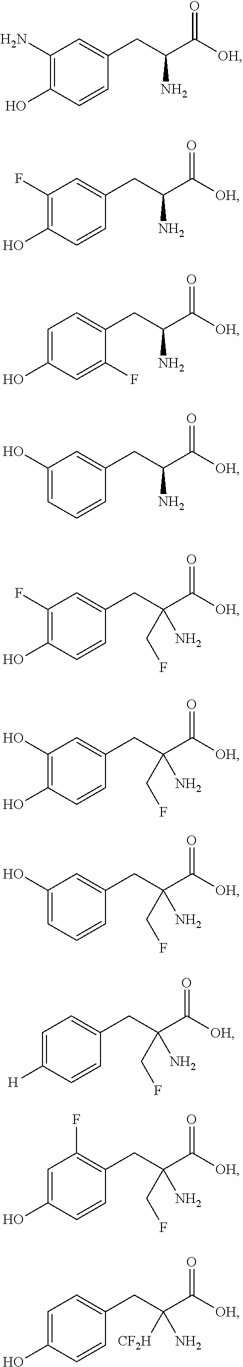 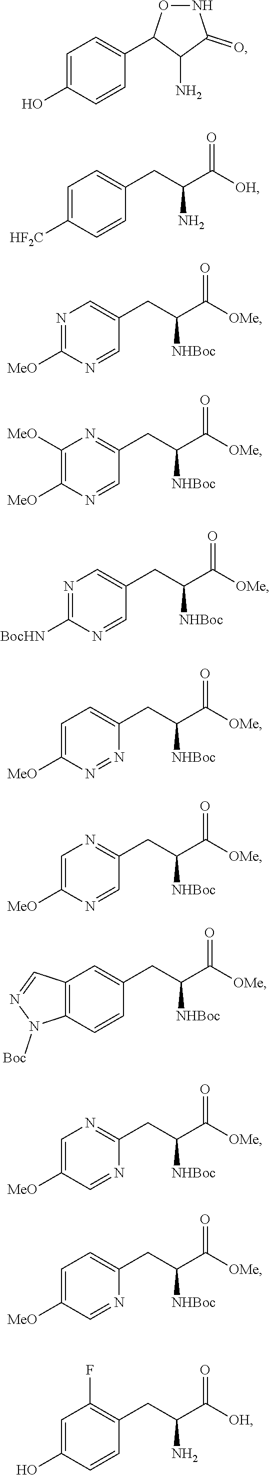 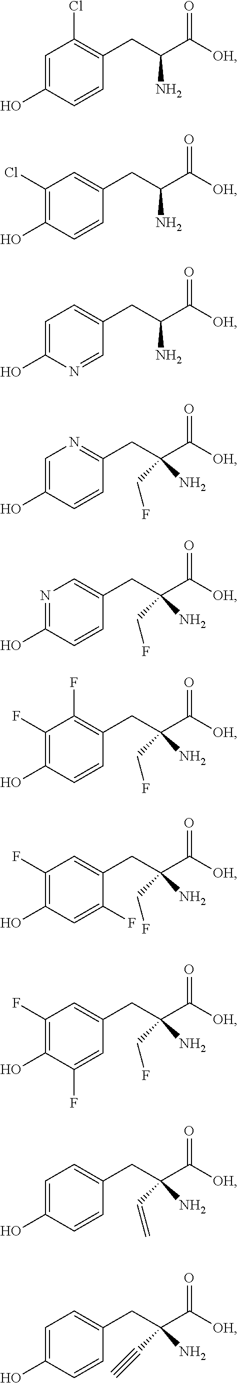 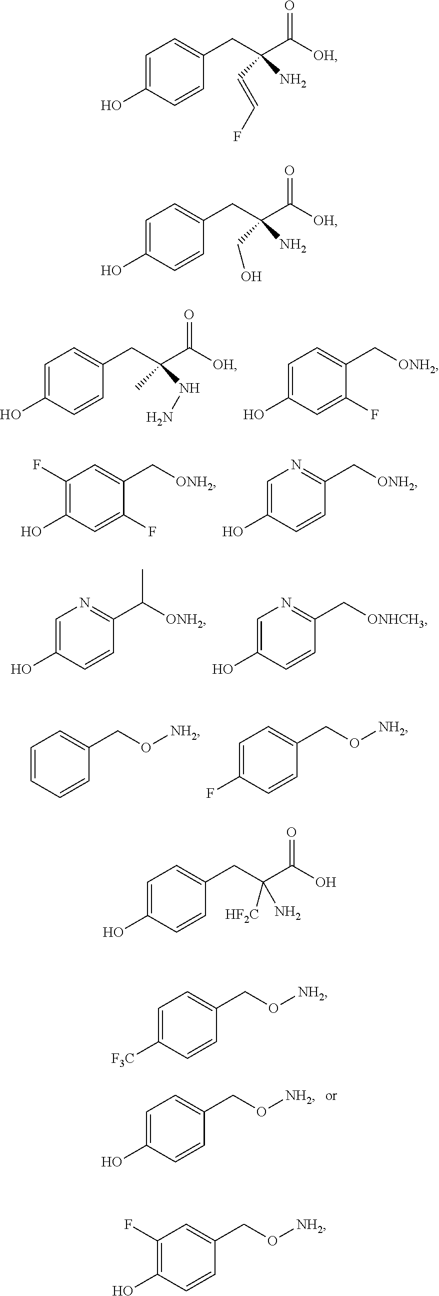
a pharmaceutically acceptable salt thereof.

14. The compound of any one of the preceding claims, wherein the compound is:

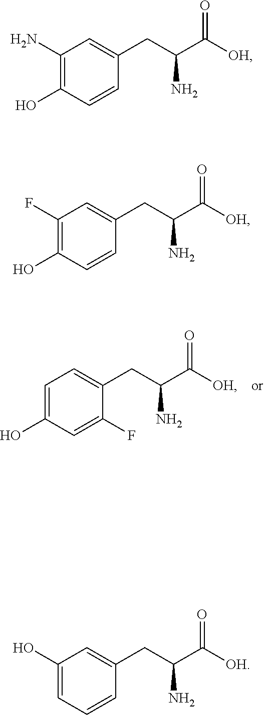 [SMILES: Nc1cc(C[C@H](N)C(=O)O)ccc1O ; N[C@@H](Cc1ccc(O)c(F)c1)C(=O)O ; N[C@@H](Cc1ccc(O)cc1F)C(=O)O ; N[C@@H](Cc1cccc(O)c1)C(=O)O]

15. The compound of any one of the preceding claims, wherein the compound is:

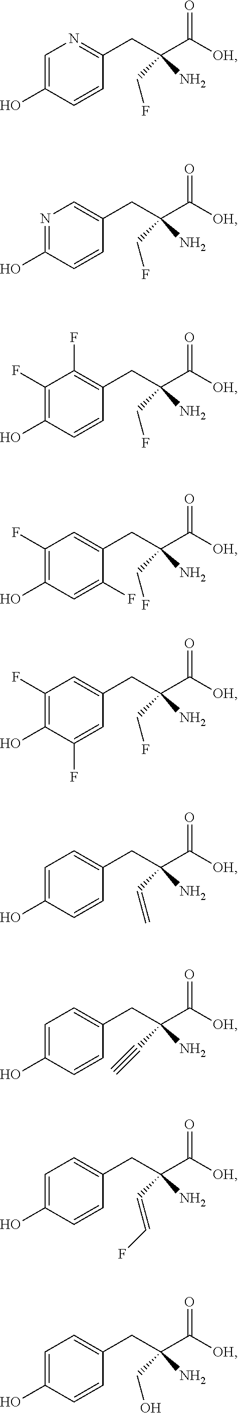

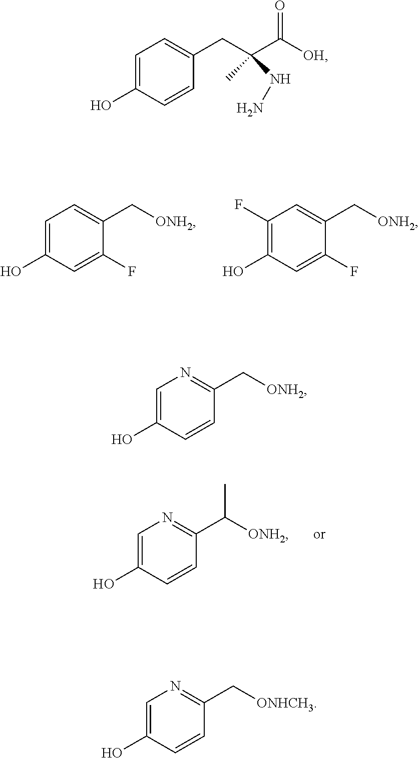

16. A method of treating Parkinson's Disease in a subject in need thereof, comprising administering to the subject a therapeutically effective amount of a compound of formula (I) or formula (II): 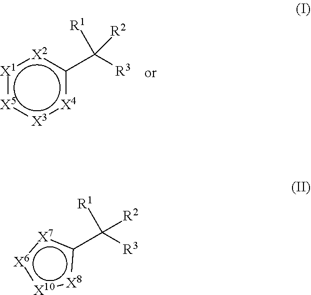
or a pharmaceutically acceptable salt thereof, wherein, as permitted by valence and stability:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or ═C(R¹¹)—;
X³ is ═N— or ═C(R′)—;
X⁴ is ═N— or ═C(R¹²)—;
provided that
no more than two of X¹ to X⁵ are ═N—; and
when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R⁸)—;
X¹⁰ is ═N—, —NR¹³—, or ═C(R⁴)—;
provided that, if at least three of X⁶ to X¹⁰ are ═N—, one of X⁶ to X¹⁰ is —NR¹³—
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂, or 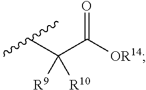
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

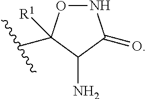

17. The method of claim 16, wherein R¹⁴ is, independently at each occurrence, H or CH₃.

18. The method of claim 16, wherein R³ is

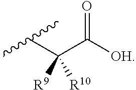

19. The method of claim 16, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or 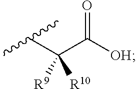
and
R¹⁰ is amino, —ONH₂, or —NHNH₂.

20. The method of any one of claims 16-19, provided the compound is not

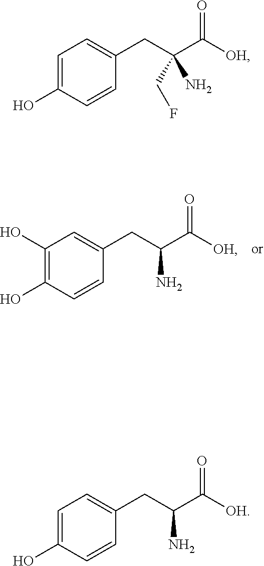 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

21. The method of claim 20, further provided that the compound is not

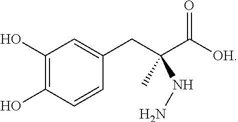 [SMILES: C[C@@](Cc1ccc(O)c(O)c1)(NN)C(=O)O]

22. The method of any one of claims 16-19, wherein the compound of formula (I) is:

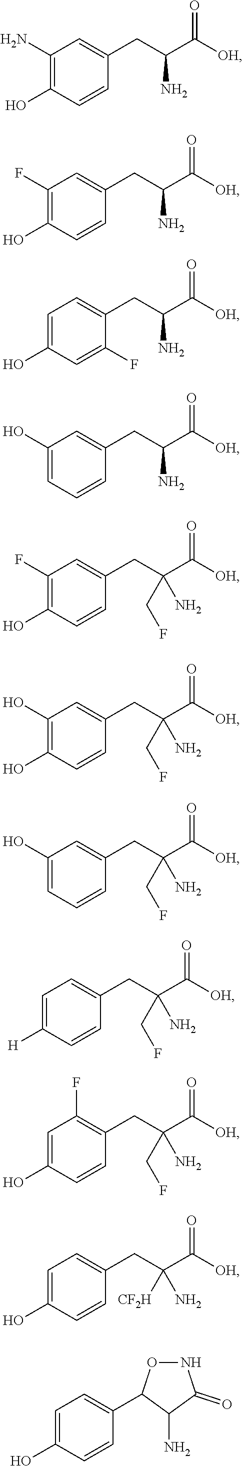

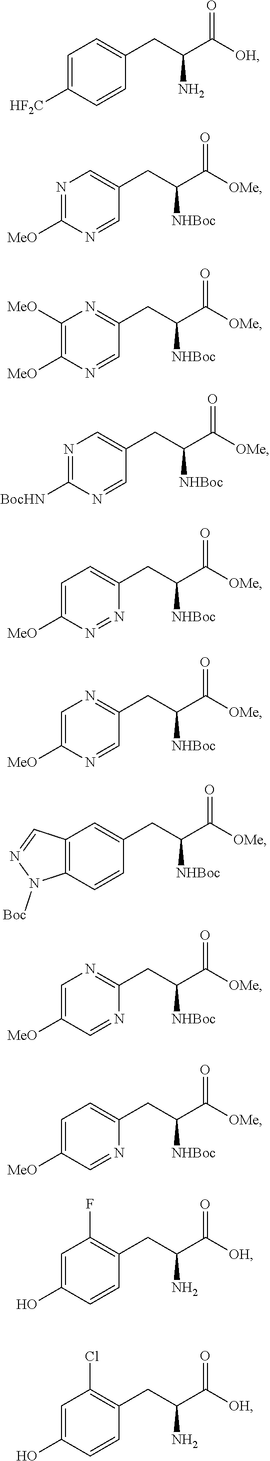

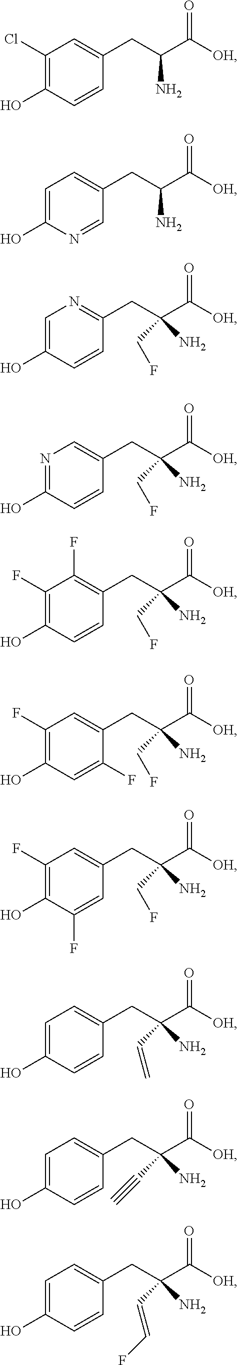

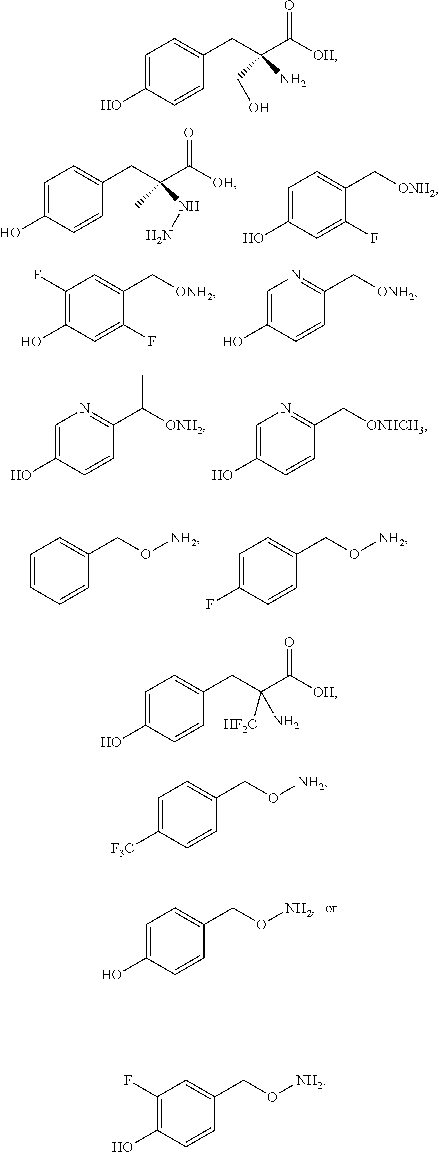

23. The method of claim 22, wherein the compound of formula (I) is 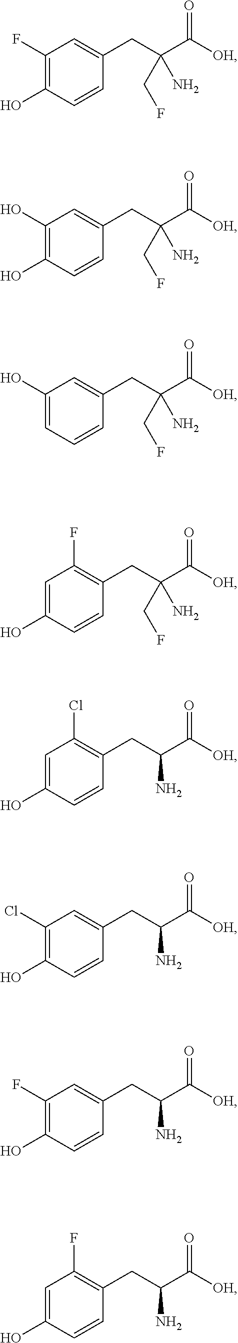 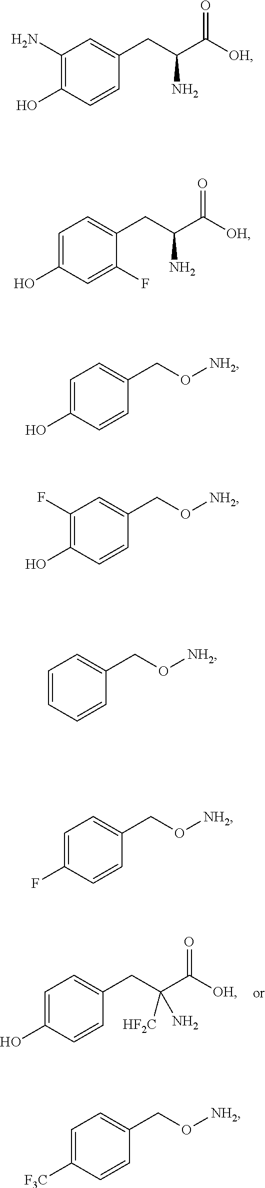
or a pharmaceutically acceptable salt thereof.

24. A method of inhibiting a tyrosine decarboxylase (TyrDC) comprising contacting the TyrDC with a compound of formula (I) or formula (II): 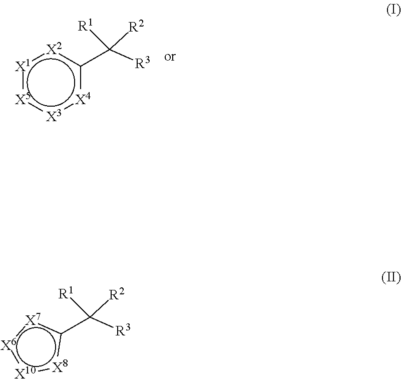
or a pharmaceutically acceptable salt thereof, wherein:
X⁵ is ═N—, —C(O)—, or ═C(R⁴)—;
R⁴ is hydroxyl; methoxy; amino; halogen; hydrogen; —N(R⁶)C(O)R⁵; or C₁₋₆ alkyl, optionally substituted with one or more halogen;
X¹ is ═N—, —N(OH)—, or —C(R⁷)═;
X² is —N═, —C(O)—, or ═C(R¹¹)—;
X³ is ═N— or ═C(R′)—;
X⁴ is ═N— or ═C(R¹²)—;
provided that
no more than two of X¹ to X⁵ are ═N—; and
when X¹ is —N(OH)—, one and only one of X² and X⁵ is —C(O)—;
X⁶ is ═N—, —NR¹³—, or ═C(R⁷)—;
X⁷ is ═N—, —NR¹³—, or ═C(R¹¹)—;
X⁸ is ═N—, —NR¹³—, or ═C(R′)—;
X¹⁰ is ═N—, —NR¹³—, or ═C(R⁴)—;
provided that, if at least three of X⁶ to X¹⁰ are ═N—, one of X⁶ to X¹⁰ is —NR¹³—
R⁶ is H or C₁₋₆ alkyl;
R⁵ is C₁₋₆ alkyl;
R¹¹ is hydrogen, halogen, C₁₋₆ alkyl, amino, or —N(R⁶)C(O)R⁵;
R¹² is hydrogen, halogen, C₁₋₆ alkyl, amino, or —N(R⁶)C(O)R⁵;
R⁷ is hydrogen, hydroxyl, amino, halogen, or —N(R⁶)C(O)R⁵;
R⁸ is hydrogen, hydroxyl, amino, halogen, or —N(R⁶)C(O)R⁵;
or R⁴ and R⁷, or R⁴ and R⁸, or R⁷ and R¹¹, or R⁸ and R¹², together with the carbon atoms to which they are attached, combine to form a 5- to 6-membered carbocyclic or heterocyclic ring;
R¹³ is hydrogen, halogen, C₁₋₆ alkyl, amino, or —N(R⁶)C(O)R⁵;
R¹ is hydrogen or C₁₋₆ alkyl;
R² is hydrogen or C₁₋₆ alkyl;
R³ is —ONR¹⁴₂, —NHNH₂, or 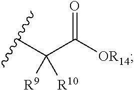
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

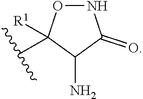

25. The method of claim 24, wherein R¹⁴ is, independently at each occurrence, H or CH₃.

26. The method of claim 24, wherein R³ is

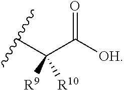

27. The method of claim 24, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or 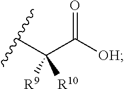
and
R¹⁰ is amino, —ONH₂, or —NHNH₂.

28. The method of any one of claims 24-27, provided the compound is not

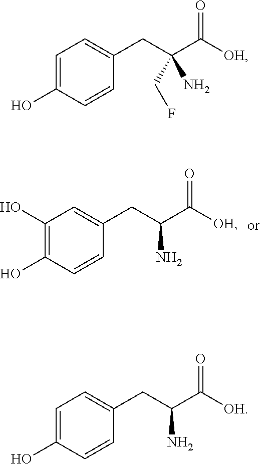 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

29. The method of claim 28, further provided that the compound is not

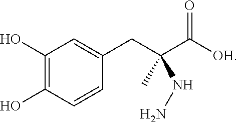 [SMILES: C[C@@](Cc1ccc(O)c(O)c1)(NN)C(=O)O]

30. The method of any one of claims 24-27, wherein the compound of formula (I) is:

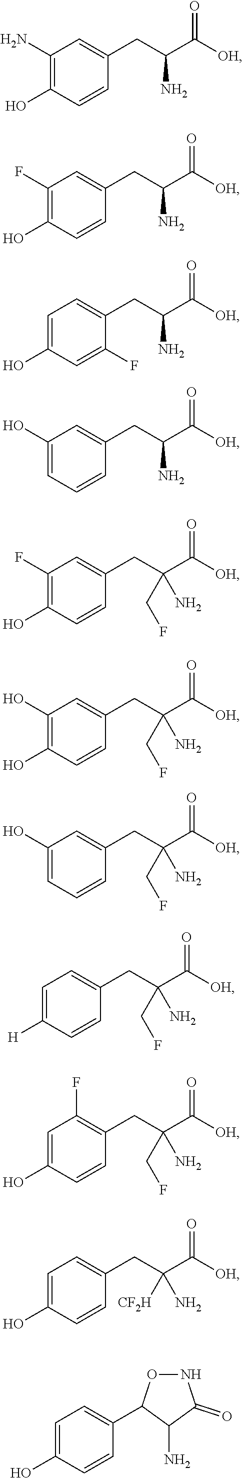

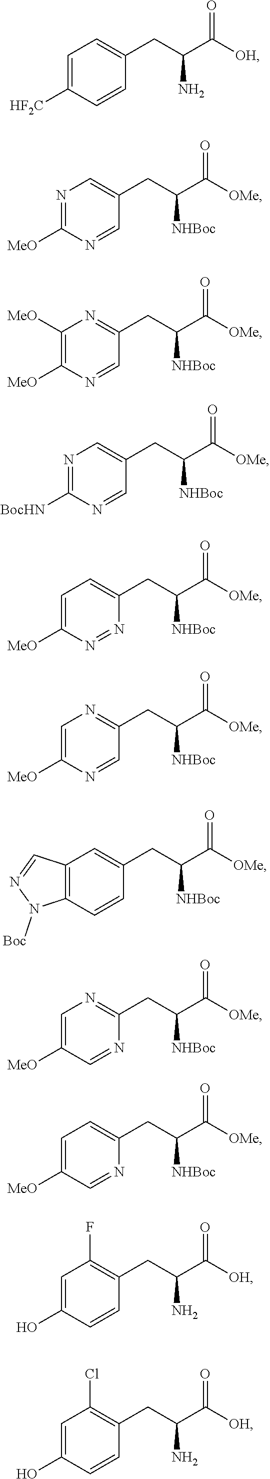

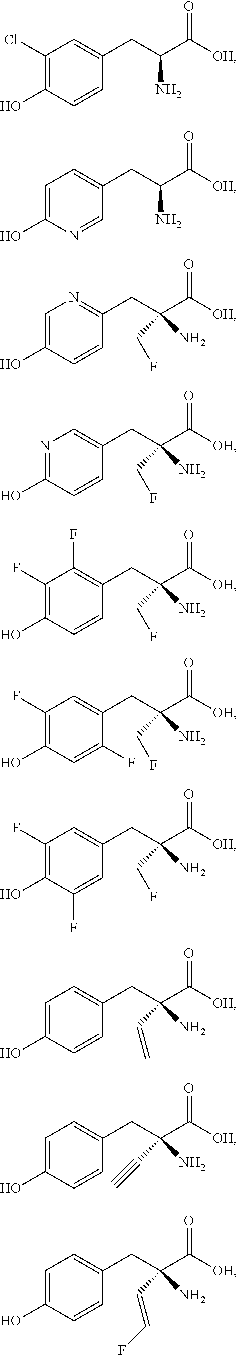

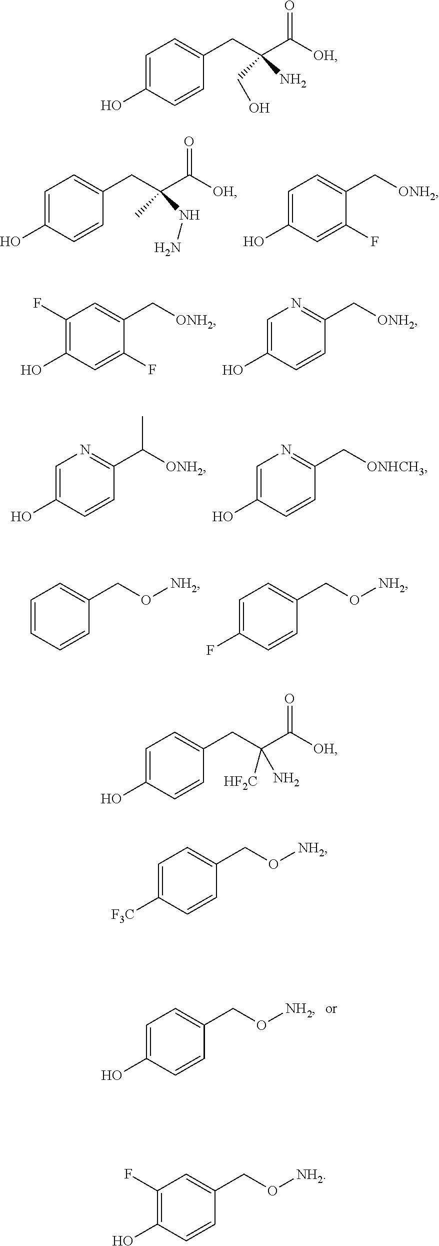

31. The method of claim 30, wherein the compound of formula (I) is 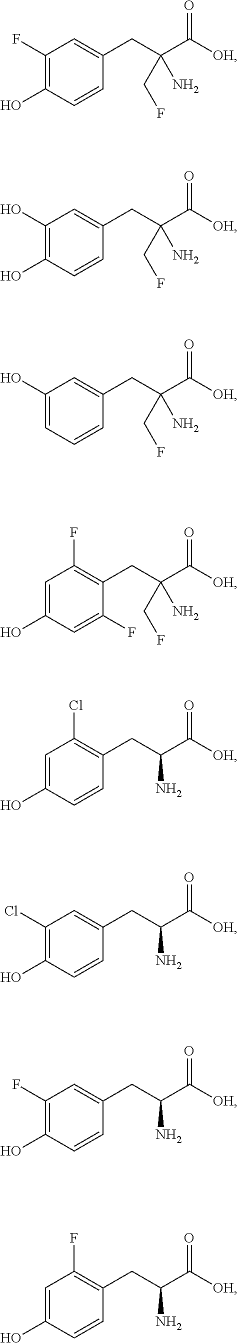 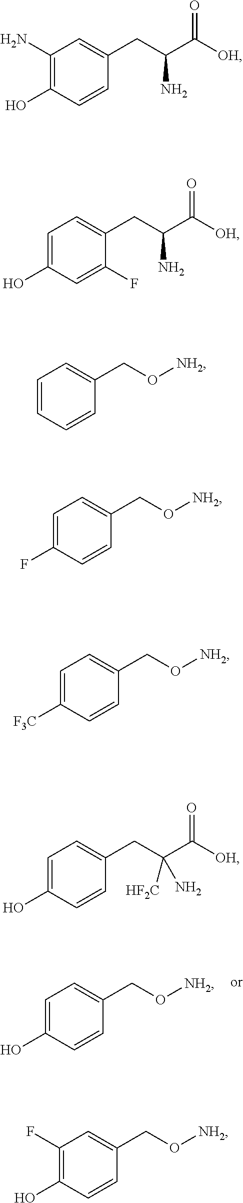
or a pharmaceutically acceptable salt thereof.

32. The method of any one of claims 24-31, wherein the TyrDC is Enterococcus faecalis TyrDC.

33. A compound of formula (Ia): 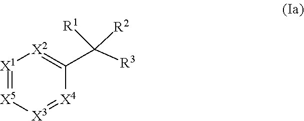
or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or 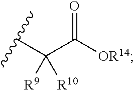
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

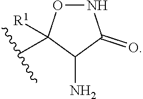

34. The compound of claim 33, wherein R¹⁴ is, independently at each occurrence, H or CH₃.

35. The compound of claim 33, wherein R³ is

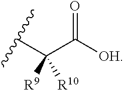

36. The compound of claim 33, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂ or 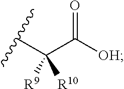
and
R¹⁰ is amino, —ONH₂, or —NHNH₂.

37. The compound of any one of claims 33-36, provided the compound is not

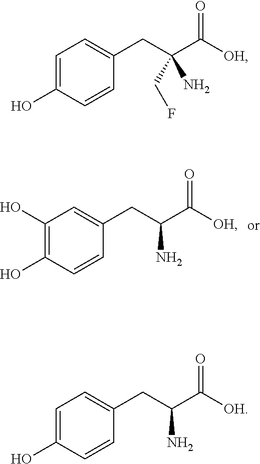 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

38. The compound of claim 37, further provided that the compound is not

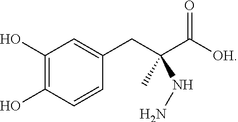 [SMILES: C[C@@](Cc1ccc(O)c(O)c1)(NN)C(=O)O]

39. The compound of any one of claims 33-38, wherein R³ is

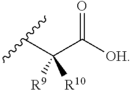

40. The compound of any one of claims 33-39, wherein X¹ is CR⁷, X² is CR¹¹, X³ is CR⁸, X⁴ is CR¹², and X⁵ is CR⁴.

41. The compound of claim 40, wherein R⁷ is H and R⁸ is H.

42. The compound of any one of claims 33-41, wherein X⁵ is CR⁴ and R⁴ is hydroxyl.

43. The compound of any one of claims 33-42, wherein R¹ is H and R² is H.

44. The compound of any one of claims 33-43, wherein R⁷ is hydroxyl, halogen, or amino.

45. The compound of any one of claims 33-44, wherein the compound is:

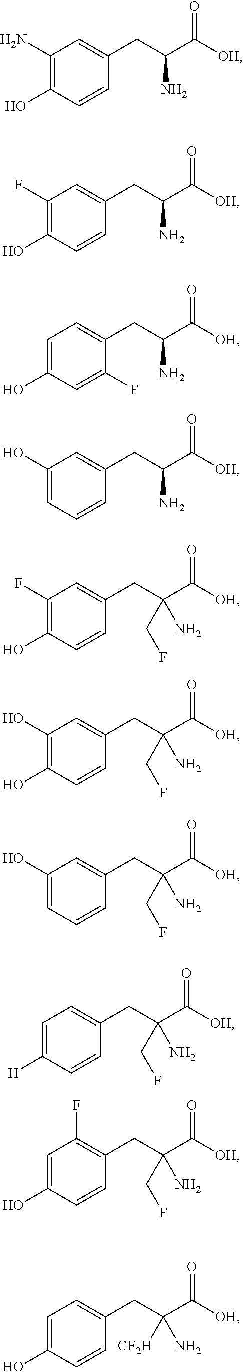

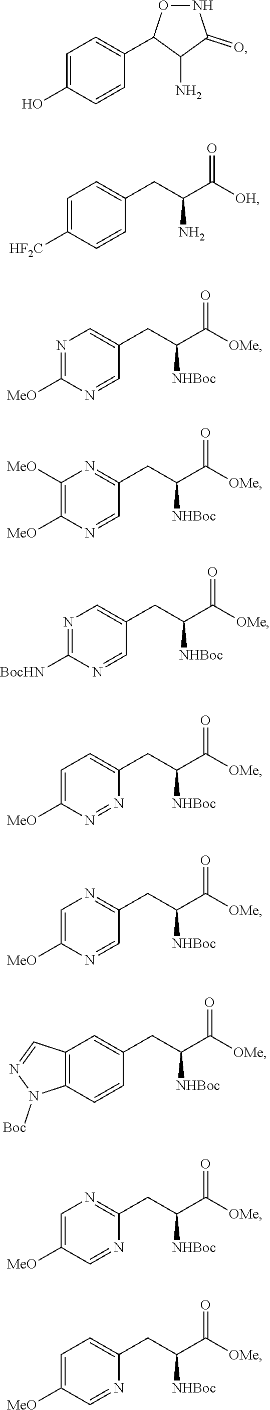

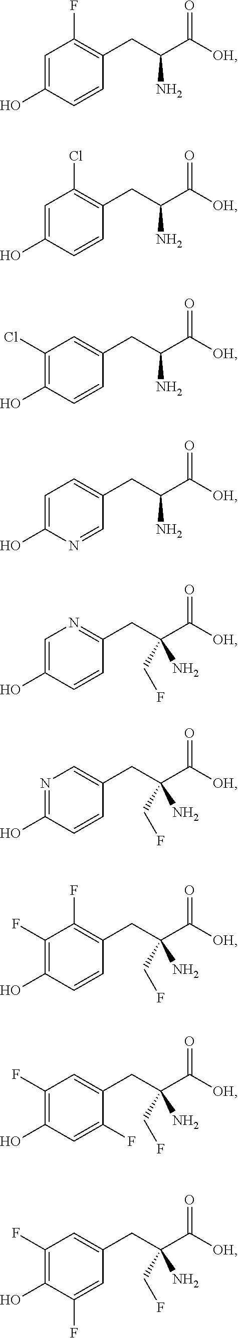

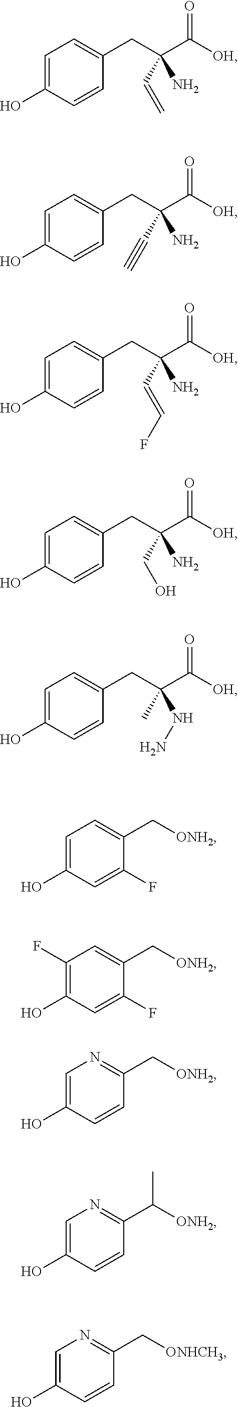

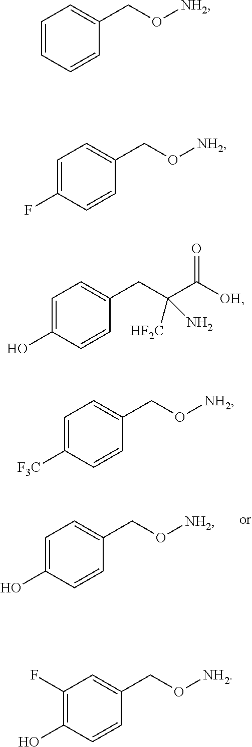

46. The compound of any one of claims 33-45, wherein the compound is:

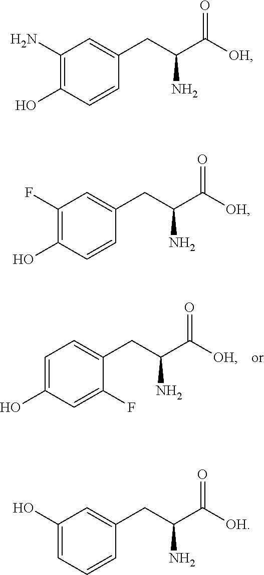 [SMILES: Nc1cc(C[C@H](N)C(=O)O)ccc1O ; N[C@@H](Cc1ccc(O)c(F)c1)C(=O)O ; N[C@@H](Cc1ccc(O)cc1F)C(=O)O ; N[C@@H](Cc1cccc(O)c1)C(=O)O]

47. The compound of claim 45, wherein the compound is:

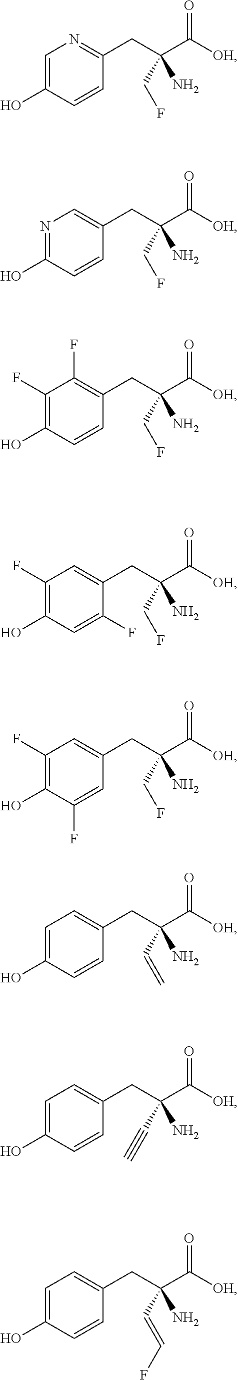

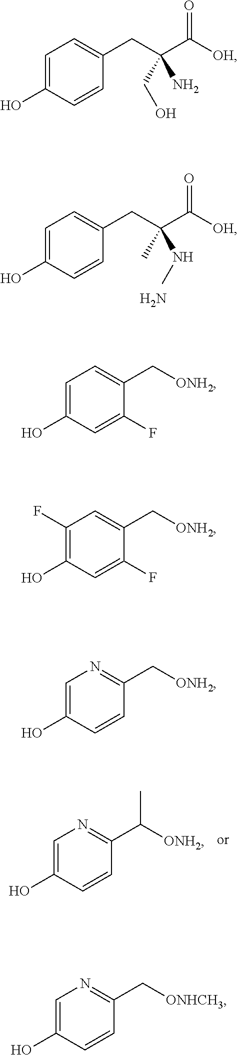

48. A method of treating Parkinson's Disease in a subject in need thereof, comprising administering to the subject a therapeutically effective amount of a compound of formula (Ia): 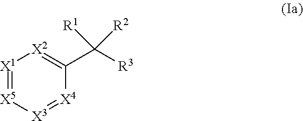
or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or 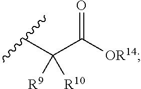
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

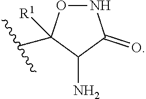

49. The method of claim 48, wherein R¹⁴ is, independently at each occurrence, H or CH₃.

50. The method of claim 48, wherein R³ is

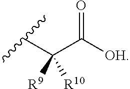

51. The method of claim 48, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is-NHNH₂, —ONH₂, or 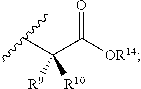
R¹⁰ is amino, —ONH₂, or —NHNH₂.

52. The method of any one of claims 48-51, provided the compound is not

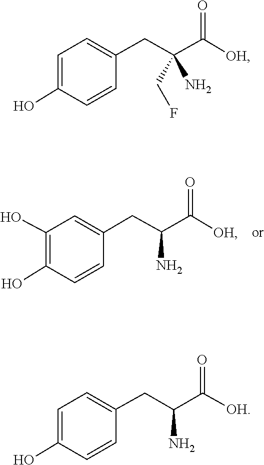 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

53. The method of claim 52, further provided that the compound is not

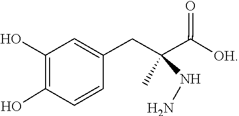 [SMILES: C[C@@](Cc1ccc(O)c(O)c1)(NN)C(=O)O]

54. The method of claim 52 or claim 53, wherein the compound of formula (Ia) is:

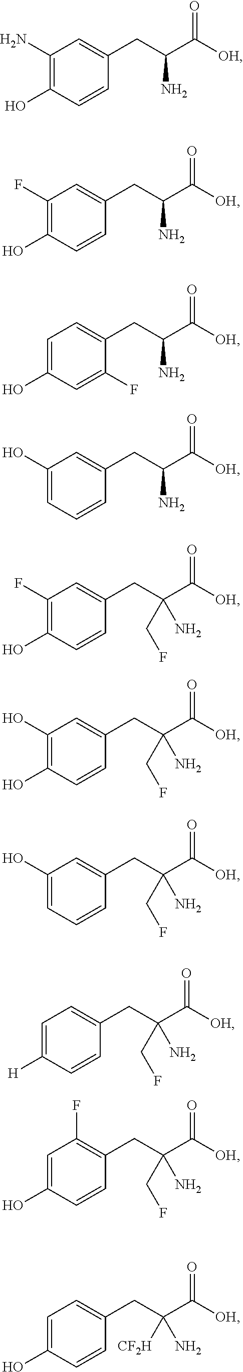

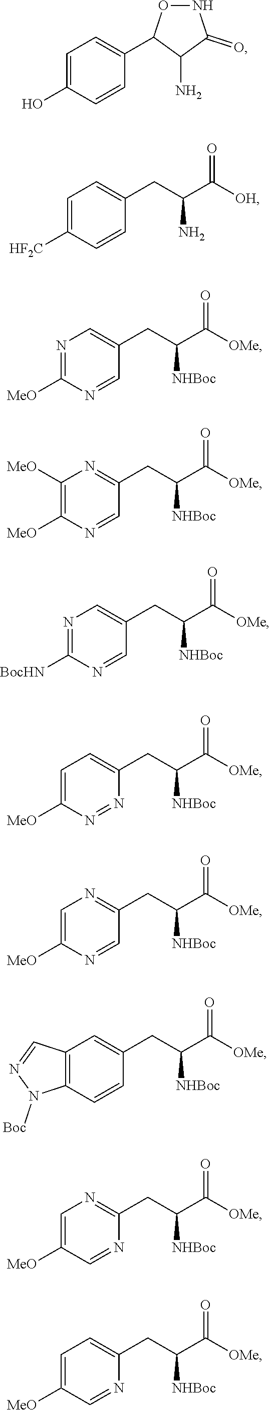

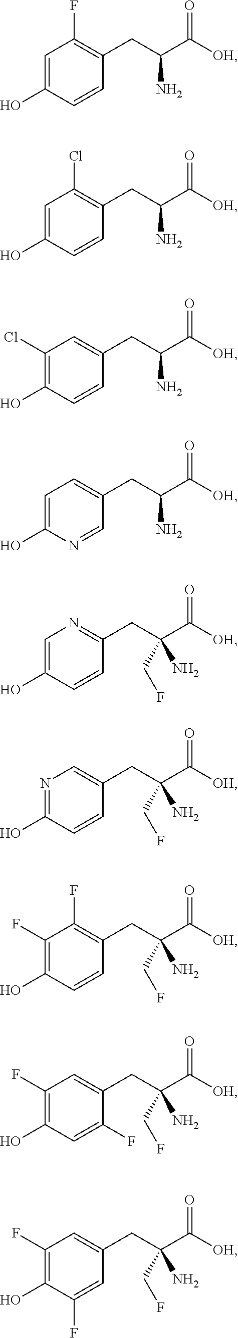

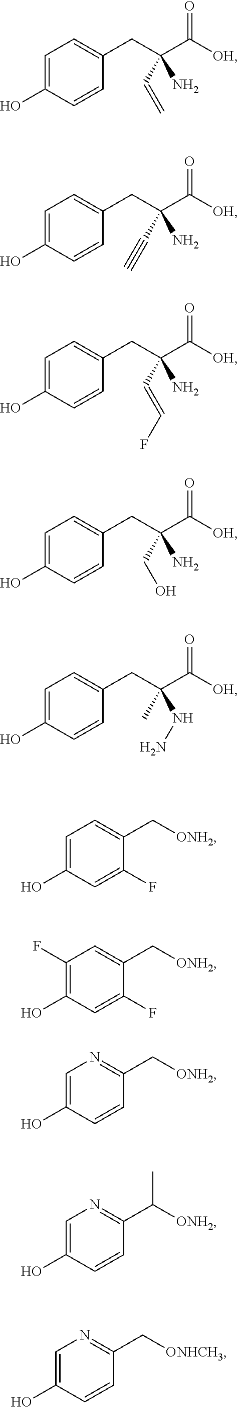

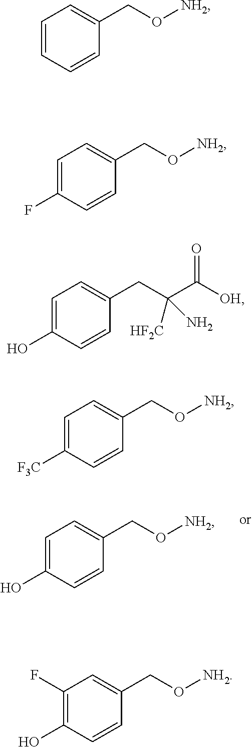

55. The method of claim 54, wherein the compound of formula (I) is 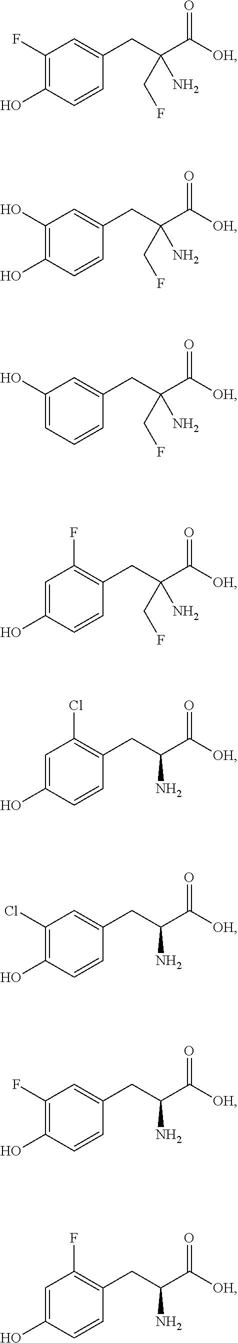 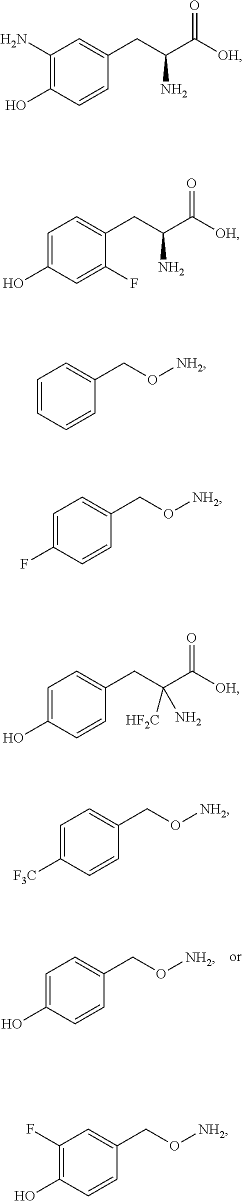
or a pharmaceutically acceptable salt thereof.

56. A method of inhibiting a tyrosine decarboxylase (TyrDC) comprising contacting the TyrDC with a compound of formula (Ia): 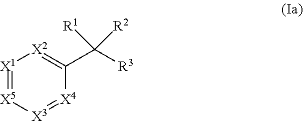
or a pharmaceutically acceptable salt thereof, wherein:
X⁵ is N or CR⁴;
R⁴ is hydroxyl; methoxy; amino; halogen; hydrogen; —N(R⁶)C(O)R⁵; or C₁₋₆ alkyl, optionally substituted with one or more halogen;
X¹ is N or CR⁷;
X² is N or CR¹¹;
X³ is N or CR⁸;
X⁴ is N or CR¹²;
provided that no more than two of X¹ to X⁵ are N;
R⁶ is H or C₁₋₆ alkyl;
R⁵ is C₁₋₆ alkyl;
R¹¹ is hydrogen, halogen, or C₁₋₆ alkyl;
R¹² is hydrogen, halogen, or C₁₋₆ alkyl;
R⁷ is hydrogen, hydroxyl, amino, or halogen;
R⁸ is hydrogen, hydroxyl, amino, or halogen;
or R⁴ and R⁷, or R⁴ and R⁸, together with the carbon atoms to which they are attached, combine to form a 5- to 6-membered carbocyclic or heterocyclic ring;
R¹ is hydrogen or C₁₋₆ alkyl;
R² is hydrogen or C₁₋₆ alkyl;
R³ is —ONR¹⁴₂, —NHNH₂, or 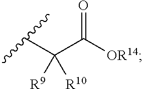
R¹⁴ is, independently at each occurrence, H or C₁₋₆ alkyl;
R⁹ is hydrogen or is C₂₋₆ alkynyl, C₂₋₆ alkenyl, C₁₋₆ alkyl, each optionally substituted with one or more halogen, hydroxyl, amino, cyano, or alkoxy; and
R¹⁰ is amino, -NHBoc, —ONH₂, or —NHNH₂;
or R² and R³, together with the carbon atom to which they are attached, combine to form

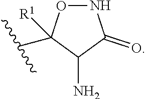

57. The method of claim 56, wherein R¹⁴ is, independently at each occurrence, H or CH₃.

58. The method ofclaim 56, wherein R³ is

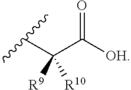

59. The method of claim 56, wherein:
R⁴ is hydroxyl; amino; halogen; hydrogen; —N(R⁶)C(O)R⁵; or C₁₋₆ alkyl, optionally substituted with one or more halogen;
R¹ is hydrogen or C₁₋₆ alkyl;
R² is hydrogen or C₁₋₆ alkyl;
R³ is —NHNH₂, —ONH₂, or 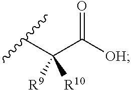
R¹⁰ is amino, —ONH₂, or —NHNH₂.

60. The method of any one of claims 56-59, provided the compound is not

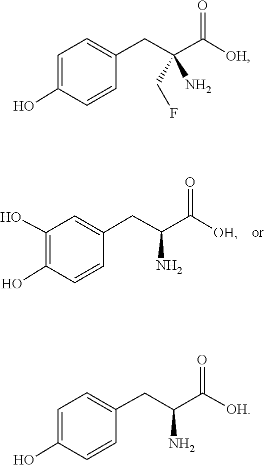 [SMILES: N[C@@](CF)(Cc1ccc(O)cc1)C(=O)O ; N[C@@H](Cc1ccc(O)c(O)c1)C(=O)O ; N[C@@H](Cc1ccc(O)cc1)C(=O)O]

61. The method of claim 60, further provided that the compound is not

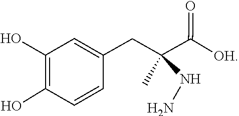 [SMILES: C[C@@](Cc1ccc(O)c(O)c1)(NN)C(=O)O]

62. The method of any one of claims 56-60, wherein the compound is

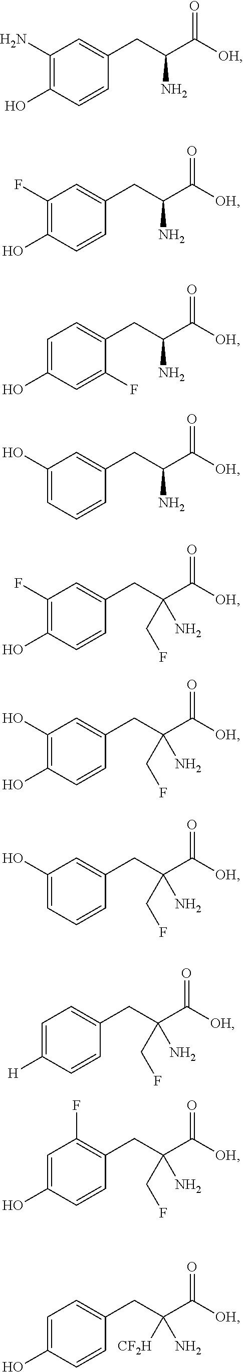

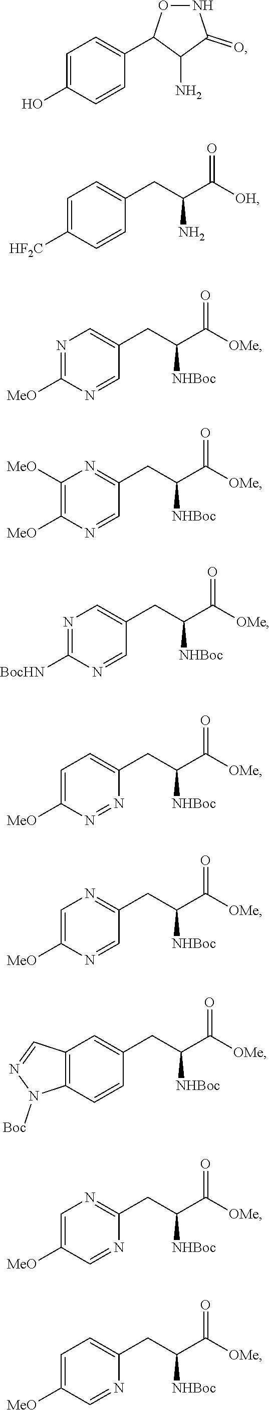

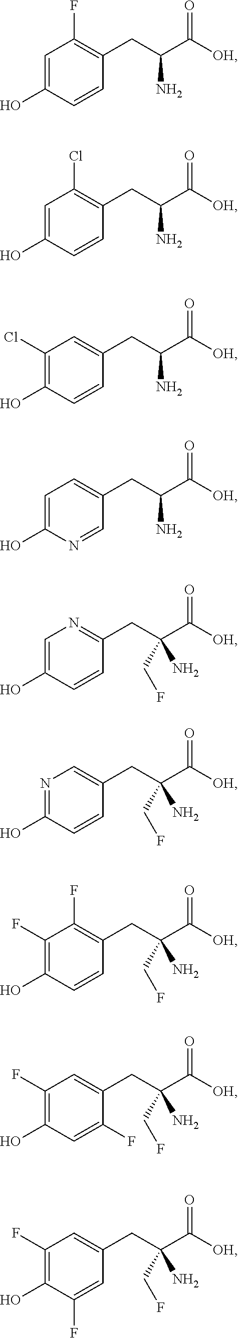

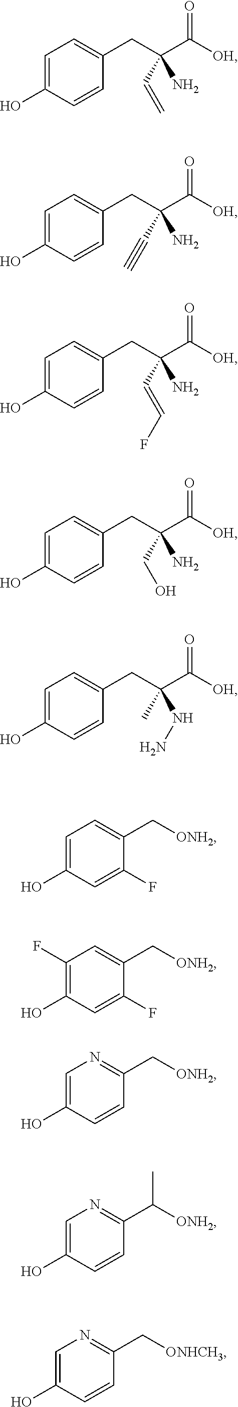

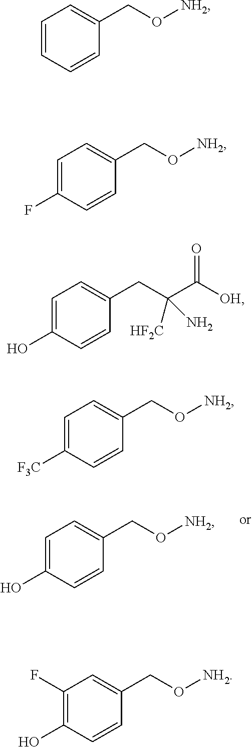

63. The method of claim 62, wherein the compound of formula (I) is 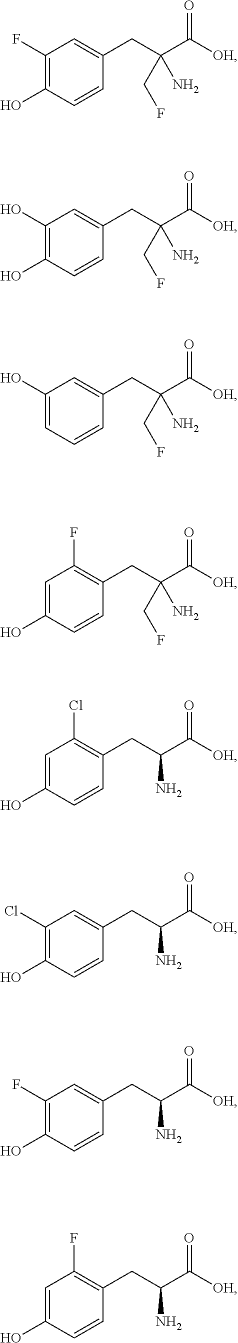 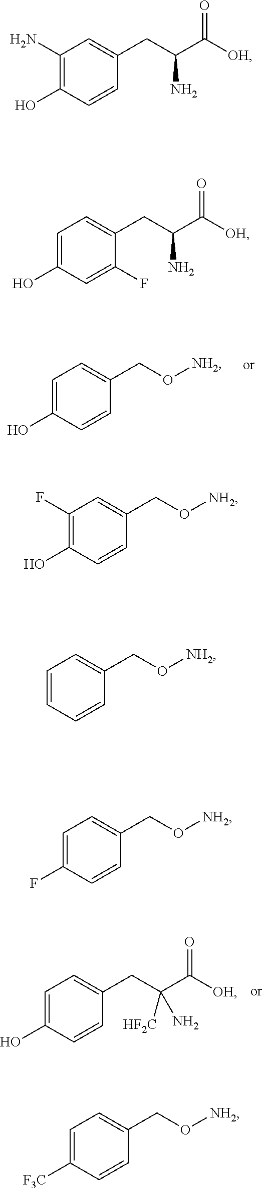
or a pharmaceutically acceptable salt thereof.

64. The method of any one of claims 56-63, wherein the TyrDC is Enterococcus faecalis TyrDC.
